(19) United States
(12) Patent Application Publication
(10) Pub. No.: US 2005/0095213 A1
(43) Pub. Date: May 5, 2005

(54) Two-coat cosmetic product, cosmetic process of using thereof and makeup kit containing this product

(76) Inventors: Xavier Blin, Paris (FR); Veronique Ferrari, Maisons-Alfort (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/949,448

(22) Filed: Sep. 27, 2004

Related U.S. Application Data
(60) Provisional application No. 60/539,353, filed on Jan. 28, 2004.

(30) Foreign Application Priority Data
Sep. 26, 2003 (FR) 03 11340

(51) Int. Cl. A61K 7/06; A61K 7/11

(52) U.S. Cl. 424/70.11

(57) ABSTRACT

The present disclosure relates to a cosmetic product comprising at least one first and at least one second composition, the first composition comprising, in a cosmetically acceptable organic liquid medium, at least one film-forming linear ethylenic block polymer, and the second composition comprising a cosmetically acceptable medium. The present disclosure also relates to a makeup process and to a makeup kit containing the product. This product can be, for example, a lipstick, a mascara or a nail varnish.

This application claims benefit of U.S. Provisional Application No. 60/539,353, filed Jan. 28, 2004.

The present disclosure relates to a cosmetic product comprising at least two compositions that can be applied successively to the skin of the face and of the body, the lower and upper eyelids, the lips and integuments such as the nails, the eyebrows, the eyelashes or the hair. The present disclosure also relates to a process for making up the face and the body using the at least two compositions.

Each composition may be a foundation, a makeup rouge, an eyeshadow, a concealer product, a blusher, a free or compacted powder, a lipstick, a lip balm, a lip gloss, a lip pencil, an eye pencil, a mascara, an eyeliner, a nail varnish or a body-makeup or skin-coloring product.

Most known cosmetic compositions have poor staying power over time, for example, poor color fastness and poor staying power of the gloss over time. This poor staying power can be characterized by a modification of the color such as color change and/or fading, which can generally follow an interaction with the sebum and/or sweat secreted by the skin in the case of foundations and makeup powders, or an interaction with the saliva in the case of lipsticks, or a reduction in the gloss, in the case of a nail varnish. This can require the user to reapply the makeup frequently, which may constitute a loss of time.

In addition, many of the known cosmetic compositions have a tendency to migrate, i.e. to travel over time and collect in the folds of the wrinkles and fine lines of the skin, such as those around the lips and the eyes, which can result in an unattractive effect. Cosmetics users often mention this migration as a major defect of cosmetic compositions, for instance, standard lipsticks and eyeshadows. The term “migration” means an overflowing or shifting of the composition and for example, of the color, beyond the initial line of the makeup.

“Transfer-resistant” lip and skin makeup compositions are compositions that can have the advantage of forming a deposit that does not always come off, at least partly, onto the supports with which they are placed in contact, for example, glass, clothing, cigarette or fabrics. The known transfer-resistant compositions are generally based on silicone resins and volatile silicone oils, and, although most have improved staying power properties, they can have the drawback of leaving on the skin and the lips, after evaporation of the volatile silicone oils, a film that becomes uncomfortable over time, creating a sensation of dryness and tautness, which discourages certain consumers from using lipsticks of this type.

In addition, these transfer-resistant compositions based on volatile silicone oils and silicone resins mostly lead to matte colored films. Now, cosmetics users are nowadays in search of products, such as for coloring the lips or the eyelids, that are glossy while at the same time capable of having good staying power and being transfer-resistant.

To obtain transfer-resistant products capable of exhibiting good staying power, volatile oils or polymers dispersed in a volatile solvent are often used. However, these products are rarely considered sufficiently glossy. A cosmetic product comprising two compositions, wherein the first composition contains a polymer dispersed in a volatile solvent, over which is applied a second glossy and greasy composition, has thus been envisaged, i.e., French Patent No.-A-2,823,101. The staying power of the color of the makeup obtained with these compositions can be good, but the disappearance of the second composition in the course of the day when it is worn can result in a loss of gloss, but the gloss is desired by the consumer.

The company Kose has moreover proposed in Japanese patent application JP-A-05,221,829 to use a gel based on perfluoro materials, which is applied over a film of lipstick so as to prevent it from being transferred onto other surfaces, the gel being incompatible with the film of lipstick.

Mention may also be made of patent application WO-A-97/17057, which describes a method for increasing the staying-power and transfer-resistance properties, comprising applying two compositions one over the other. The composition to be applied as a basecoat has a global Hildebrand solubility parameter of less than 8.5 (cal/cm³)^{1/2}, and the composition to be applied as a topcoat contains oils whose calculated coefficient of partition ClogP is at least equal to 13.

U.S. Pat. No. 6,001,374 proposes a multicoat makeup system, which comprises using a composition containing a resin that is soluble in alcohol and insoluble in water, which may be applied as a basecoat or as a topcoat, and which can have the advantage of not marking an object placed in contact with the makeup and of being resistant to water and to rubbing, while at the same time having a certain level of gloss. However, this composition contains a water-soluble alcohol, specifically ethanol, which has an irritant, dehydrating nature on the skin and more especially on the lips, and which can be uncomfortable when the skin or the lips are damaged.

Patent application WO 02/067877 describes a method for improving the aesthetic properties of a transfer-resistant composition, which comprises applying a second composition over a film of transfer-resistant composition. The second composition should not interact chemically with the transfer-resistant composition, so as not to impair its cosmetic properties. Some of the products described in the document have an unpleasant odor and are tacky. Other products are not glossy enough.

Thus, there is a need for a novel route for formulating a cosmetic product, such as a makeup product, of the type comprising two compositions to be applied successively one after the other. Accordingly, one aspect of the present disclosure is, for example, to prepare a cosmetic product that allows good transfer-resistance and/or gloss fastness properties, compared for instance, with the product of patent application WO-A-97/17057. Another aspect of the present disclosure is to propose a cosmetic product, such as a makeup product, that can simultaneously combine at least some of the following properties: transfer resistance, migration resistance, colorfastness, comfort, absence of dehydration, gloss and staying power of the gloss over time.

Thus, one aspect of the present disclosure is combining at least one first composition comprising, in a cosmetically acceptable organic liquid medium, at least one film-forming linear ethylenic block polymer, and at least one second composition comprising a cosmetically acceptable medium. Therefore, the compositions according to at least some of the aspects of the present disclosure make it possible to obtain a cosmetic result that can be very glossy on application and over time, which does not migrate, does not transfer and/or can show good staying power, while at the same time being comfortable on application and over time, i.e., non-tacky, non-dehydrating, and no tautness.

For example, the product of the present disclosure makes it possible to obtain non-tacky continuous deposits on the skin or the lips, which can give good coverage, can have a very glossy appearance, which is adapted to the desire of consumers, rarely migrates, rarely transfers, can have good staying power, is not oily and rarely dries out the skin, the hair or the lips onto which it is applied, either during application or over time. It can also have good stability properties and thus allow a uniform and aesthetic application.

It has moreover been found that the compositions of the product according to the present disclosure can have certain advantageous spreading and adhesion properties on the skin, the lips or the eyelashes, and also a creamy and pleasant feel.

These staying-power, transfer-resistant and migration-resistant properties, allied with the glossy and non-greasy appearance, can make a product that is suitable for producing makeup products for the lips such as lipstick and lip glosses, for the eyes, such as mascara, eyeliners and eyeshadows, and for the nails or the hair.

One aspect of the present disclosure is thus a cosmetic product for application to the skin, for instance, the skin of the face or the neck, the lips or the eyelids, comprising at least one first and at least one second composition, wherein the at least one first composition comprises, in a cosmetically acceptable organic liquid medium, at least one film-forming linear ethylenic block polymer, and the at least one second composition, which is different from the first, comprises a cosmetically acceptable medium.

In one embodiment of the present disclosure, the block polymer is free of styrene units.

In another embodiment of the present disclosure, the block polymer is non-elastomeric.

Another aspect of the present disclosure is a cosmetic product for application to the integuments, such as the nails, the hair or the eyebrows, comprising at least one first and at least one second composition, wherein the at least one first composition comprises, in a cosmetically acceptable organic liquid medium, at least one film-forming linear ethylenic block polymer, and the at least one second composition, which is separate from the first, comprises a cosmetically acceptable medium.

The product of the present disclosure can be for example, a makeup product for the skin, the nails or the hair. The term “makeup product” as used herein means a product comprising a coloring agent that allows the deposition of a color onto a keratin material such as the skin or the integuments, of a human being by applying onto the keratin material products such as lipsticks, makeup powders, eyeliners, foundations, self-tanning agents or semi-permanent makeup products (tattoos).

The product according to the present disclosure comprises at least two cosmetically acceptable compositions packaged separately or together in the same packaging article or in at least two separate packaging articles. For example, these compositions can be packaged separately and, in one embodiment of the present disclosure, the compositions are packaged in separate packaging articles.

One aspect of the present disclosure is thus, for instance, a cosmetic makeup product in the form of a mascara, a makeup rouge, an eyeshadow, a lipstick, a nail varnish, a product, for example, having care properties, a mascara, an eyeliner, a concealer product or a makeup product for the body (such as a tattoo) or the hair.

Another aspect of the present disclosure is a makeup kit comprising a cosmetic makeup product as defined above, wherein the various compositions are packaged separately and are for instance, accompanied by suitable application means. These application means may be fine brushes, coarse brushes, pens, pencils, felts, quills, sponges, tubes and/or foam tips.

The at least one first composition of the product according to the present disclosure may constitute a base coat applied onto the keratin material, and the at least one second composition may constitute a topcoat. It is possible, however, to apply under the first coat an undercoat that may or may not have the constitution of the second coat.

It is also possible to deposit an overcoat onto the second coat, which may or may not have an identical constitution to that of the first coat. In one embodiment of the present disclosure, the makeup obtained is a two-coat makeup.

The at least one second composition may also constitute a basecoat applied onto the keratin material, and the at least one first composition a topcoat. For example, in another embodiment of the present disclosure, the base coat is a lipstick, a mascara, a lip gloss, an eyeliner, a nail varnish, a nailcare product or a body makeup product, and the top coat is a care or protective product.

The present disclosure also relates to a process for making up the skin and/or the lips and/or the integuments, which comprises applying a cosmetic product as defined above to the skin and/or the lips and/or the integuments.

Still another aspect of the present disclosure is a cosmetic care or makeup process for human skin and/or lips and/or integuments, which comprises applying to the skin, the lips and/or the integuments a first coat of at least one first composition comprising, in a cosmetically acceptable organic liquid medium, at least one film-forming linear ethylenic block polymer, and then in applying, over all or part of the at least one first coat, at least one second coat of a second composition comprising a cosmetically acceptable medium. This process may comprise applying to human skin, lips and/or integuments a first coat of at least one first composition comprising, in a cosmetically acceptable organic liquid medium, at least one film-forming linear ethylenic block polymer, leaving the at least one first coat to dry, and then applying, over all or part of the at least one first coat, at least one second coat of a second composition comprising a cosmetically acceptable medium. This process may also comprise applying to human skin, lips and/or integuments at least one first coat of a composition comprising a cosmetically acceptable medium, leaving the first coat to dry, and then applying, over all or part of the at least one first coat, at least one second coat of a composition comprising, in a cosmetically acceptable organic liquid medium, at least one film-forming linear ethylenic block polymer.

The product according to the present disclosure may be applied, not only to the face, but also the scalp and the body, the lips, the inside of the lower eyelids, and integuments, for instance the nails, the eyelashes, the hair, the eyebrows, or other body hairs. The at least one second composition may form units and may be applied with a pen, a pencil or any other instrument such as a sponge, finger, fine brush, coarse brush or quill. This makeup may also be applied to makeup accessories, for instance false nails, false eyelashes, wigs or pastilles or patches that adhere to the skin or the lips, such as beauty spots.

The present disclosure also relates to a made-up support, comprising at least one first coat of at least one first composition comprising a cosmetically acceptable first medium, and at least one second coat of at least one second composition comprising a cosmetically acceptable second medium, wherein the first coat being applied to the support first or over all or part of the second coat. This support may be, for example, a hairpiece such as a wig, false nails, false eyelashes, or patches that adhere to the skin or the lips (such as beauty spots).

The present disclosure further relates to the cosmetic use of the cosmetic product defined above to improve the comfort properties, for instance, in terms of the absence of tack and/or absence of dehydration, and/or the gloss and/or the transfer and/or the migration and/or the staying power of the makeup on the skin and/or the lips and/or the integuments.

Still another aspect of the present disclosure is the use of a cosmetic product comprising at least one first and at least one second composition, the at least one first composition comprising, in a cosmetically acceptable organic liquid medium, at least one film-forming linear ethylenic block polymer as described herein, and the at least one second composition comprising a cosmetically acceptable medium, to give the skin and/or the lips and/or the integuments a comfortable and/or glossy and/or transfer-resistant and/or migration-resistant cosmetic result and/or a cosmetic result with good staying power.

Block Polymer of the First Composition

The block polymer of the first composition of the product according to the present disclosure may be, for example, a film-forming linear ethylenic block polymer.

The first composition comprises at least one block polymer. The term “block polymer” as used herein, means a polymer comprising at least two different blocks, for example at least three different blocks.

According to one embodiment of the present disclosure, the block polymer of the first composition is an ethylenic polymer. The term “ethylenic polymer” as used herein means a polymer obtained by polymerizing monomers comprising an ethylenic unsaturation.

According to another embodiment of the present disclosure, the block polymer of the first composition is a linear polymer. In contrast, a polymer of non-linear structure is, for example, a polymer of branched, starburst or grafted structure, or the like.

According to yet another embodiment of the present disclosure, the block polymer of the first composition is a film-forming polymer. The term “film-forming polymer” as used herein, means a polymer capable of forming, by itself or in the presence of an auxiliary film-forming agent, a continuous film that adheres to a support, such as keratin materials.

According to still another embodiment of the present disclosure, the block polymer of the first composition is a non-elastomeric polymer. The term “non-elastomeric polymer” as used herein, means a polymer which, when it is subjected to a constraint intended to stretch it, for example by 30% relative to its initial length, does not return to a length substantially identical to its initial length when the constraint ceases.

For example, the term “non-elastomeric polymer” denotes a polymer with an instantaneous recovery Rᵢ<50% and a delayed recovery R₂ₕ<70% after having been subjected to a 30% elongation. For example, Rᵢ is <30% and R₂ₕ<50%.

For instance, the non-elastomeric nature of the polymer is determined according to the following protocol:

A polymer film is prepared by pouring a solution of the polymer in a Teflon-coated mold, followed by drying for seven days in an environment conditioned at 23±5° C. and 50±10% relative humidity.

A film about 100 μm thick is thus obtained, from which are cut rectangular specimens, for example using a punch, 15 mm wide and 80 mm long. The sample is subjected to a tensile stress using a machine sold under the reference Zwick, under the same temperature and humidity conditions as for the drying. The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e., about 0.3 times its initial length (l₀)
the constraint is released by applying a return speed equal to the tensile speed, i.e., 50 mm/min, and the residual elongation of the specimen is measured as a percentage, after returning to zero constraint (ε₁).

The percentage instantaneous recovery (Rᵢ) is given by the following formula:
Rᵢ=(εₘₐₓ−ε₁)/εₘₐₓ)×100

To determine the delayed recovery, the percentage residual elongation of the specimen (ε₂ₕ) is measured 2 hours after returning to zero constraint.

The percentage delayed recovery (R₂ₕ) is given by the following formula:
R₂ₕ=(εₘₐₓ−ε₂ₕ)/εₘₐₓ)×100

Purely as a guide, a polymer according to one embodiment of the present disclosure can have an instantaneous recovery Rᵢ of 10% and a delayed recovery R₂ₕ of 30%.

According to another embodiment of the present disclosure, the block polymer of the first composition does not comprise any styrene units. Thus, as used herein, the expression “polymer free of styrene units” means a polymer comprising less than 10%, such as less than 5%, for instance less than 2% and less than 1% by weight: i) of styrene units of formula —CH(C₆H₅)—CH₂— or ii) of substituted styrene units, for instance methylstyrene, chlorostyrene or chloromethylstyrene.

According to yet another embodiment of the present disclosure, the block polymer of the first composition is derived from aliphatic ethylenic monomers. The term “aliphatic monomer” means a monomer comprising no aromatic groups.

According to still another embodiment of the present disclosure, the block polymer is an ethylenic polymer derived from aliphatic ethylenic monomers comprising a carbon-carbon double bond and at least one ester group —COO— or amide group —CON—. The ester group may be linked to one of the two unsaturated carbons via the carbon atom or the oxygen atom. The amide group may be linked to one of the two unsaturated carbons via the carbon atom or the nitrogen atom.

Further according to another embodiment, the block polymer comprises at least one first block and at least one second block. The term “at least one block” means one or more blocks.

It is pointed out that, in the text hereinabove and hereinbelow, the terms “first” and “second” blocks do not in any way condition the order of the said blocks in the polymer structure.

According to one embodiment of the present disclosure, the block polymer comprises at least one first block and at least one second block that have different glass transition temperatures (Tg). In this embodiment, the first and second blocks may be linked together via an intermediate segment with a glass transition temperature between the glass transition temperatures of the first and second blocks.

According to another embodiment, the block polymer comprises at least one first block and at least one second block linked together via an intermediate segment comprising at least one constituent monomer of the first block and at least one constituent monomer of the second block. For example, the intermediate block is derived essentially from constituent monomers of the first block and of the second block. The term “essentially” means at least 85%, preferably at least 90%, better still 95% and even better still 100%.

For example, the intermediate segment comprising at least one constituent monomer of the first block and at least one constituent monomer of the second block of the polymer may be a random polymer.

According to yet another embodiment, the block polymer comprises at least one first block and at least one second block that are incompatible in the organic liquid medium of the composition of the invention. Thus, the phrase “mutually incompatible blocks” means that the mixture formed from the polymer corresponding to the first block and of the polymer corresponding to the second block is not miscible in the organic liquid that is in major amount by weight contained in the organic liquid medium of the composition, at room temperature (25° C.) and atmospheric pressure (10⁵ Pa), for an amount of the polymer mixture of greater than or equal to 5% by weight, relative to the total weight of the mixture (polymers and major organic liquid), it being understood that:
i) the polymers are present in the mixture in an amount such that the respective weight ratio ranges from 10/90 to 90/10, and
ii) each of the polymers corresponding to the first and second blocks has an average (weight-average or number-average) molecular mass equal to that of the block polymer±15%.

When the organic liquid medium comprises a mixture of organic liquids, in the case when at least two liquids are present in identical mass proportions, the said polymer mixture is immiscible in at least one of them. When the organic liquid medium comprises only one organic liquid, this liquid obviously constitutes the liquid that is in major amount by weight. As used herein, the term “organic liquid medium” means a medium comprising at least one organic liquid, i.e., at least one organic compound that is liquid at room temperature (25° C.) and atmospheric pressure (10⁵ Pa). According to one embodiment of the present disclosure, the major liquid of the organic liquid medium is a volatile or non-volatile oil (fatty substance). For example, the organic liquid is cosmetically acceptable, that is acceptable tolerance, toxicology and feel.

According to another embodiment, the major liquid of the organic liquid medium is at least one of the polymerization solvents of the block polymer. As used herein, the term “polymerization solvent” means a solvent or a mixture of solvents. The polymerization solvent may be chosen for example, from ethyl acetate, butyl acetate, alcohols such as isopropanol and ethanol, aliphatic alkanes such as isododecane, and mixtures thereof. For instance, the polymerization solvent can be a mixture of butyl acetate and isopropanol, or isododecane.

In general, the block polymer may be incorporated into the composition at a high solids content, for example, greater than 10%, greater than 20%, for instance, greater than 30% and greater than 45% by weight relative to the total weight of the composition, while at the same time being easy to formulate.

In one embodiment of the present disclosure, the block polymer comprises no silicon atoms in its skeleton. The term “skeleton” means the main chain of the polymer, as opposed to the pendent side chains.

For example, the polymer according to the present disclosure can be water-insoluble, i.e. the polymer is not soluble in water or in a mixture of water and linear or branched lower monoalcohols comprising from 2 to 5 carbon atoms, for instance ethanol, isopropanol or n-propanol, without pH modification, at an active material content of at least 1% by weight, at room temperature (25° C.).

According to another embodiment, the block polymer has a polydispersity index I of greater than 2.

For example, the block polymer used in the compositions according to the present disclosure can have a polydispersity index I of greater than 2, for example ranging from 2 to 9, such as greater than or equal to 2.5, for example ranging from 2.5 to 8 and such as greater than or equal to 2.8, for instance, ranging from 2.8 to 6.

The polydispersity index I of the polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector).

The weight-average mass (Mw) of the block polymer may be, for example, less than or equal to 300,000; it can range, for example, from 35000 to 200,000, such as from 45,000 to 150,000.

The number-average mass (Mn) of the block polymer may be, for example, less than or equal to 70,000; it can range, for example, from 10,000 to 60,000, for instance from 12,000 to 50,000.

Each block of the block polymer can be derived from one type of monomer or from several different types of monomer. This means that each block may comprise a homopolymer or a copolymer; this copolymer comprising the block may in turn be random or alternating.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$1 / Tg = {\sum }_{i} ( {ϖ}_{i} / T {g}_{i} ) ,$$

wherein \overline{ω}ᵢ is the mass fraction of the monomer i in the block under consideration and Tgᵢ is the glass transition temperature of the homopolymer of the monomer i. Unless otherwise indicated, the Tg values indicated for the first and second blocks in the present patent application are theoretical Tg values.

The difference between the glass transition temperatures of the first and second blocks is generally greater than 10° C., such as greater than 20° C. and for instance greater than 30° C. For example, the block polymer can comprise at least one first block and at least one second block such that the first block may be chosen from:
a) a block with a Tg of greater than or equal to 40° C.,
b) a block with a Tg of less than or equal to 20° C.,
c) a block with a Tg of between 20 and 40° C.,
   and the second block can be chosen from a category a), b) or c) different from the first block.

In the present disclosure, the expression:“between . . . and . . . ” is intended to denote a range of values for which the limits mentioned are excluded, and “from . . . to . . . ” and “ranging from . . . to . . . ” are intended to denote a range of values for which the limits are included.

a) Block with a Tg of greater than or equal to 40° C.

The block with a Tg of greater than or equal to 40° C. has, for example, a Tg ranging from 40 to 150° C., for instance a Tg greater than or equal to 50° C., for example ranging from 50° C. to 120° C., such as greater than or equal to 60° C., for example ranging from 60° C. to 120° C. The block with a Tg of greater than or equal to 40° C. can be a homopolymer or a copolymer. The block with a Tg of greater than or equal to 40° C. can be totally or partially derived from at least one monomer, which is such that the homopolymer prepared from these monomers has a glass transition temperature of greater than or equal to 40° C.

In the case where this block is a homopolymer, it is derived from monomers which are such that the homopolymers prepared from these monomers have glass transition temperatures of greater than or equal to 40° C. This first block may be a homopolymer comprising only one type of monomer (for which the Tg of the corresponding homopolymer is greater than or equal to 40° C).

In the case where the first block is a copolymer, it can be totally or partially derived from at least one monomers, the nature and concentration of which is chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40 to 150° C., such as greater than or equal to 50° C., for example ranging from 50° C. to 120° C., such as greater than or equal to 60° C., for example ranging from 60° C. to 120° C., and
monomers which are such that the homopolymers prepared from these monomers have Tg values of less than 40° C., chosen from monomers with a Tg ranging from 20 to 40° C. and/or monomers with a Tg of less than or equal to 20° C., for example a Tg ranging from −100 to 20° C., such as less than 15° C., for instance ranging from −80° C. to 15° C., such as less than 10° C., for example ranging from −50° C. to 0° C., as described herein.

Among the monomers whose homopolymers have a glass transition temperature of greater than or equal to 40° C., non-limiting mention may be made of:
methacrylates of formula CH₂═C(CH₃)—COOR₁
   wherein R₁ is chosen from linear and branched unsubstituted alkyl groups comprising from 1 to 4 carbon atoms, such as a methyl, ethyl, propyl or isobutyl group, and C₄ to C₁₂ cycloalkyl groups,
acrylates of formula CH₂═CH—COOR₂
   wherein R₂ is chosen from C₄ to C₁₂ cycloalkyl groups such as isobornyl acrylate or a tert-butyl group,
(meth)acrylamides of formula: 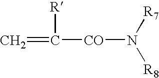
   wherein R₇ and R₈, which may be identical or different, are chosen from hydrogen atoms and linear and branched C₁ to C₁₂ alkyl group such as an n-butyl, t-butyl, isopropyl, isohexyl, isooctyl or isononyl group; or R₇ is hydrogen and R₈ is 1,1-dimethyl-3-oxobutyl,
   and R′ is chosen from a hydrogen atom and methyl radicals. Non-limiting examples of monomers that may be mentioned include N-butylacrylamide, N-t-butylacrylamide, N-isopropylacrylamide, N,N-dimethylacrylamide and N,N-dibutylacrylamide,
and mixtures thereof.

For example, in one embodiment of the present disclosure, the monomers are chosen from methyl methacrylate, isobutyl (meth)acrylate, isobornyl (meth)acrylate, and mixtures thereof.

b) Block with a Tg of less than or equal to 20° C.

The block with a Tg of less than or equal to 20° C. has, for example, a Tg ranging from −100 to 20° C., such as a Tg less than or equal to 15° C., for instance ranging from −80° C. to 15° C., such as a Tg less than or equal to 10° C., for example ranging from −50° C. to 0° C. The block with a Tg of less than or equal to 20° C. can be a homopolymer or a copolymer. The block with a Tg of less than or equal to 20° C. can be totally or partially derived from at least one monomer, which are such that the homopolymer prepared from the at least one monomer has a glass transition temperature of less than or equal to 20° C. In the case where this block is a homopolymer, it is derived from monomers which are such that the homopolymers prepared from these monomers have glass transition temperatures of less than or equal to 20° C. This second block may be a homopolymer comprising only one type of monomer, for which the Tg of the corresponding homopolymer is less than or equal to 20° C.

In the case where the block with a Tg of less than or equal to 20° C. is a copolymer, it may be totally or partially derived from at least one monomer, the nature and concentration of which is chosen such that the Tg of the resulting copolymer is less than or equal to 20° C.

Among the monomers that may be used, non-limiting mention may be made of, for example:
at least one monomers whose corresponding homopolymer has a Tg of less than or equal to 20° C., for example a Tg ranging from −100° C. to 20° C., such as a Tg less than 15° C., for instance ranging from −80° C. to 15° C., such as a Tg less than 10° C., for example ranging from −50° C. to 0° C., and
at least one monomer whose corresponding homopolymer has a Tg of greater than 20° C., such as monomers with a Tg of greater than or equal to 40° C., for example a Tg ranging from 40° C. to 150° C., such as a Tg greater than or equal to 50° C., for example ranging from 50° C. to 120° C., such as a Tg greater than or equal to 60° C., for example ranging from 60° C. to 120° C. and/or monomers with a Tg ranging from 20° C. and 40° C., as described above.

In one embodiment of the present disclosure, the block with a Tg of less than or equal to 20° C. is a homopolymer.

Among the monomers whose homopolymer has a Tg of less than or equal to 20° C., non-limiting mention can be made of the following monomers:
acrylates of formula CH₂═CHCOOR₃,
   wherein R₃ is chosen from linear and branched C₁ to C₁₂ unsubstituted alkyl groups, with the exception of tert-butyl groups, wherein at least one hetero atom chosen from O, N and S atoms is optionally intercalated in said alkyl groups,
methacrylates of formula CH₂═C(CH₃)—COOR₄,
   wherein R₄ is chosen from linear and branched C₆ to C₁₂ unsubstituted alkyl groups, wherein at least one hetero atom chosen from O, N and S atoms is optionally intercalated;
vinyl esters of formula R₅—CO—O—CH═CH₂
   wherein R₅ is chosen from linear and branched C₄ to C₁₂ alkyl groups,
C₄ to C₁₂ alkyl vinyl ethers,
—N—(C₄ to C₁₂)alkyl acrylamides, such as N-octylacrylamide,
and mixtures thereof.

In one embodiment of the present disclosure, the at least one monomer used for the block with a Tg of less than or equal to 20° C. is chosen from alkyl acrylates whose alkyl chain comprises from 1 to 10 carbon atoms and wherein said alkyl is not a tert-butyl group, such as methyl acrylate, isobutyl acrylate, 2-ethylhexyl acrylate, and mixtures thereof.

c) Block with a Tg of between 20° C. and 40° C.

The block with a Tg of between 20° C. and 40° C. can be a homopolymer or a copolymer. The block with a Tg of between 20° C. and 40° C. can be totally or partially derived from at least one monomer, which is such that the homopolymer prepared from these monomers has a glass transition temperature of between 20° C. and 40° C. The block with a Tg of between 20° C. and 40° C. can be totally or partially derived from monomers, which are such that the corresponding homopolymer has a Tg of greater than or equal to 40° C. and from monomers which are such that the corresponding homopolymer has a Tg of less than or equal to 20° C.

In the case where this block is a homopolymer, it is derived from at least one monomer, which is such that the homopolymers prepared from the at least one monomer have glass transition temperatures of between 20° C. and 40° C. This first block may be a homopolymer, comprising only one type of monomer, for which the Tg of the corresponding homopolymer ranges from 20° C. to 40° C.

The monomers whose homopolymer has a glass transition temperature of between 20° C. and 40° C. may be chosen from, for example, n-butyl methacrylate, cyclodecyl acrylate, neopentyl acrylate, isodecylacrylamide, and mixtures thereof.

In the case where the block with a Tg of between 20° C. and 40° C. is a copolymer, it is totally or partially derived from at least one monomer whose nature and concentration is chosen such that the Tg of the resulting copolymer is between 20° C. and 40° C.

By way of non-limiting example, the block with a Tg of between 20° C. and 40° C. may be a copolymer totally or partially derived from:
monomers whose corresponding homopolymer has a Tg of greater than or equal to 40° C., for example a Tg ranging from 40° C. to 150° C., such as a Tg greater than or equal to 50° C., for example ranging from 50° C. to 120° C., such as a Tg greater than or equal to 60° C., for example ranging from 60° C. to 120° C., as described above, and/or
monomers whose corresponding homopolymer has a Tg of less than or equal to 20° C., for example a Tg ranging from −100 to 20° C., such as a Tg less than or equal to 15° C., for instance ranging from −80° C. to 15° C., such as a Tg less than or equal to 10° C., for example ranging from −50° C. to 0° C., as described above,
   the monomers being chosen such that the Tg of the copolymer forming the first block is between 20° C. and 40° C.

Non-limiting examples of such monomers include, for example, from methyl methacrylate, isobornyl acrylate and methacrylate, butyl acrylate, 2-ethylhexyl acrylate, and mixtures thereof.

For example, the proportion of the second block with a Tg of less than or equal to 20° C. ranges from 10% to 85% by weight, for instance from 20% to 70% and from 20% to 50% by weight, relative to the total weight of the polymer.

In one embodiment, each of the first and second blocks comprises at least one monomer chosen from acrylic acid, acrylic acid esters, methacrylic acid, methacrylic acid esters, and mixtures thereof. For example, each of the first and second blocks is totally derived from at least one monomer chosen from acrylic acid, acrylic acid esters, methacrylic acid, methacrylic acid esters, and mixtures thereof. However, each of the blocks may comprise in small proportion at least one constituent monomer of the other block.

Thus, the first block may comprise at least one constituent monomer of the second block, and vice versa.

Each of the first and/or second blocks may comprise, in addition to the monomers indicated above, at least one additional monomer which is different from the monomers mentioned above. The nature and amount of the at least one additional monomer is chosen such that the block in which they are present has the desired glass transition temperature.

This at least one additional monomer is chosen, for example, from:
a) hydrophilic monomers such as:
   ethylenically unsaturated monomers comprising at least one carboxylic or sulfonic acid function, for instance:
   acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof,
   ethylenically unsaturated monomers comprising at least one tertiary amine functional group, for instance 2-vinylpyridine, 4-vinylpyridine, dimethylaminoethyl methacrylate, diethylaminoethyl methacrylate and dimethylaminopropylmethacrylamide, and salts thereof,
   methacrylates of formula CH₂═C(CH₃)—COOR₆
      wherein R₆ is chosen from linear and branched alkyl groups comprising from 1 to 4 carbon atoms, such as a methyl, ethyl, propyl or isobutyl groups, the said alkyl groups being substituted with at least one substituent chosen from hydroxyl groups (for instance 2-hydroxypropyl methacrylate and 2-hydroxyethyl methacrylate) and halogens (such as Cl, Br, I or F), such as trifluoroethyl methacrylate,
   methacrylates of formula CH₂═C(CH₃)—COOR₉,
      wherein R₉ is chosen from linear and branched C₆ to C₁₂ alkyl groups in which at least one hetero atom chosen from O, N and S atoms is optionally intercalated, the alkyl group being substituted with at least one substituent chosen from hydroxyl groups and halogens (such as Cl, Br, I or F);
   acrylates of formula CH₂═CHCOOR₁₀,
      wherein R₁₀ is chosen from linear and branched C₁ to C₁₂ alkyl group substituted with at least one substituent chosen from hydroxyl groups and halogens (such as Cl, Br, I or F), such as 2-hydroxypropyl acrylate and 2-hydroxyethyl acrylate, or R₁₀ is chosen from C₁ to C₁₂ alkyl-O-POE (polyoxyethylene) groups with repetition of the oxyethylene unit in an amount ranging from 5 to 30 times, for example methoxy-POE, or
   R₁₀ is chosen from polyoxyethylenated groups comprising from 5 to 30 ethylene oxide units
b) ethylenically unsaturated monomers comprising at least one silicon atom, such as methacryloxypropyltrimethoxysilane and
   methacryloxypropyltris(trimethylsiloxy)silane,
   and mixtures thereof.

For example, the at least one additional monomer can be chosen from acrylic acid, methacrylic acid, trifluoroethyl methacrylate, and mixtures thereof.

According to one embodiment of the present disclosure, each of the first and second blocks of the block polymer comprises at least one monomer chosen from (meth)acrylic acid esters and optionally at least one additional monomer such as (meth)acrylic acid, and mixtures thereof.

According to another embodiment of the present disclosure, each of the first and second blocks of the block polymer is totally derived from at least one monomer chosen from (meth)acrylic acid esters and optionally at least one additional monomer such as (meth)acrylic acid, and mixtures thereof.

According to still another embodiment of the present disclosure, the block polymer is a non-silicone polymer, i.e. a polymer free of silicon atoms.

The at least one additional monomer is present in an amount of less than or equal to 30% by weight, for example ranging from 1% to 30% by weight, such as from 5% to 20% by weight, and for instance from 7% to 15% by weight, relative to the total weight of the first and/or second blocks.

The block polymer may be obtained by free-radical solution polymerization according to the following preparation process:
a portion of the polymerization solvent is introduced into a suitable reactor and heated until the adequate temperature for the polymerization is reached (typically a temperature ranging from 60° C. to 120° C.),
once this temperature is reached, the constituent monomers of the first block are introduced in the presence of some of the polymerization initiator,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.
   First Embodiment

According to one aspect of the present disclosure, the block polymer comprises a first block with a Tg of greater than or equal to 40° C., as described above in a) and a second block with a Tg of less than or equal to 20° C., as described above in b).

For example, the first block with a Tg of greater than or equal to 40° C. can be a copolymer derived from monomers which are such that the homopolymer prepared from these monomers has a glass transition temperature of greater than or equal to 40° C., such as the monomers described above.

For further example, the second block with a Tg of less than or equal to 20° C. is a homopolymer derived from monomers, which are such that the homopolymer prepared from these monomers has a glass transition temperature of less than or equal to 20° C., such as the monomers described above.

For instance, the proportion of the block with a Tg of greater than or equal to 40° C. can range from 20% to 90%, such as from 30% to 80% and from 50% to 70% by weight of the polymer. The proportion of the block with a Tg of less than or equal to 20° C. can further range from 5% to 75%, such as from 15% to 50% and from 25% to 45% by weight, relative to the total weight of the polymer.

Thus, according to a first variant, the polymer according to the present disclosure comprises:
a first block with a Tg of greater than or equal to 40° C., for example having a Tg ranging from 70° C. to 110° C., which is a methyl methacrylate/acrylic acid copolymer,
a second block with a Tg of less than or equal to 20° C., for example ranging from 0° C. to 20° C., which is a methyl acrylate homopolymer, and
an intermediate block which is a methyl methacrylate/acrylic acid/methyl acrylate copolymer.

According to a second variant, the polymer according to the present disclosure comprises:
a first block with a Tg of greater than or equal to 40° C., for example ranging from 70° C. to 100° C., which is a methyl methacrylate/acrylic acid/trifluoroethyl methacrylate copolymer,
a second block with a Tg of less than or equal to 20° C., for example ranging from 0° C. to 20° C., which is a methyl acrylate homopolymer, and
an intermediate block which is a methyl methacrylate/acrylic acid/methyl acrylate/trifluoroethyl methacrylate random copolymer.

According to a third variant, the polymer according to the present disclosure comprises:
a first block with a Tg of greater than or equal to 40° C., for example ranging from 85° C. to 115° C., which is an isobornyl acrylate/isobutyl methacrylate copolymer,
a second block with a Tg of less than or equal to 20° C., for example ranging from −85° C. to −55° C., which is a 2-ethylhexyl acrylate homopolymer, and
an intermediate block, which is an isobornyl acrylate/isobutyl methacrylate/2-ethylhexyl acrylate random copolymer.

According to a fourth variant, the polymer according to the present disclosure comprises:
a first block with a Tg of greater than or equal to 40° C., for example ranging from 85° C. to 115° C., which is an isobornyl acrylate/methyl methacrylate copolymer,
a second block with a Tg of less than or equal to 20° C., for example ranging from −85° C. to −55° C., which is a 2-ethylhexyl acrylate homopolymer, and
an intermediate block which is an isobornyl acrylate/methyl methacrylate/2-ethylhexyl acrylate random copolymer.

According to a fifth variant, the polymer according to the present disclosure comprises:
a first block with a Tg of greater than or equal to 40° C., for example ranging from 95° C. to 125° C., which is an isobornyl acrylate/isobornyl methacrylate copolymer,
a second block with a Tg of less than or equal to 20° C., for example ranging from −85° C. to −55° C., which is a 2-ethylhexyl acrylate homopolymer, and
an intermediate block which is an isobornyl acrylate/isobornyl methacrylate/2-ethylhexyl acrylate random copolymer.

According to a sixth variant, the polymer according to the present disclosure comprises:
a first block with a Tg of greater than or equal to 40° C., for example ranging from 85° C. to 115° C., which is an isobornyl methacrylate/isobutyl methacrylate copolymer,
a second block with a Tg of less than or equal to 20° C., for example ranging from −35° C. to −5° C., which is an isobutyl acrylate homopolymer, and
an intermediate block which is an isobornyl methacrylate/isobutyl methacrylate/isobutyl acrylate random copolymer.

According to a seventh variant, the polymer according to the present disclosure comprises:
a first block with a Tg of greater than or equal to 40° C., for example ranging from 95° C. to 125° C., which is an isobornyl acrylate/isobornyl methacrylate copolymer,
a second block with a Tg of less than or equal to 20° C., for example ranging from −35° C. to −5° C., which is an isobutyl acrylate homopolymer, and
an intermediate block which is an isobornyl acrylate/isobornyl methacrylate/isobutyl acrylate random copolymer.

According to an eighth variant, the polymer according to the present disclosure comprises:
a first block with a Tg of greater than or equal to 40° C., for example ranging from 60° C. to 90° C., which is an isobornyl acrylate/isobornyl methacrylate copolymer,
a second block with a Tg of less than or equal to 20° C., for example ranging from −35° C. to −5° C., which is an isobutyl acrylate homopolymer, and
an intermediate block which is an isobornyl acrylate/isobutyl methacrylate/isobutyl acrylate random copolymer.

The examples that follow illustrate, in a non-limiting manner, polymers corresponding to this first embodiment.

The amounts are expressed in grams.

### EXAMPLE 1

### Preparation of a Poly(methyl methacrylate)/acrylic acid/methyl acrylate) Polymer

100 g of butyl acetate were introduced into a 1 liter reactor and the temperature is then raised so as to pass from room temperature (25° C.) to 90° C. in 1 hour. 180 g of methyl methacrylate, 30 g of acrylic acid, 40 g of butyl acetate, 70 g of isopropanol and 1.8 g of 2,5-bis(2-ethylhexanoylperoxy)-2,5-dimethylhexane (Trigonox® 141 from Akzo Nobel) were then added.

The mixture was maintained at 90° C. for 1 hour.

90 g of methyl acrylate, 70 g of butyl acetate, 20 g of isopropanol and 1.2 g of 2,5-bis(2-ethylhexanoylperoxy)-2,5-dimethylhexane were then introduced into the above mixture, still at 90° C. and over 1 hour.

The mixture was maintained at 90° C. for 3 hours and then diluted with 105 g of butyl acetate and 45 g of isopropanol, and the mixture was then cooled.

A solution containing 40% polymer active material in a butyl acetate/isopropanol mixture was obtained.

A polymer comprising a poly(methyl methacrylate/acrylic acid) first block with a Tg of 100° C., a polymethyl acrylate second block with a Tg of 10° C. and an intermediate block which is a methyl methacrylate/acrylic acid/polymethyl acrylate random polymer was obtained.

This polymer has a weight-average mass of 52,000 and a number-average mass of 18,000, i.e., a polydispersity index I of 2.89.

### EXAMPLE 2

### Preparation of a Poly(isobornyl Acrylate/Isobutyl Methacrylate/2-ethylhexyl Acrylate) Polymer

100 g of isododecane were introduced into a 1 liter reactor and the temperature was then increased so as to pass from room temperature (25° C.) to 90° C. over 1 hour. 120 g of isobornyl acrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) were then added, at 90° C. and over 1 hour.

The mixture was maintained at 90° C. for 1 hour 30 minutes.

90 g of 2-ethylhexyl acrylate, 90 g of isododecane and 1.2 g of 2,5-bis(2-ethylhexanoylperoxy)-2,5-dimethylhexane were then introduced into the above mixture, still at 90° C. and over 30 minutes.

The mixture was maintained at 90° C. for 3 hours and was then cooled.

A solution containing 50% polymer active material in isododecane was obtained.

A polymer comprising a poly(isobornyl acrylate/isobutyl methacrylate) first block with a Tg of 80° C., a poly-2-ethylhexyl acrylate second block with a Tg of −70° C. and an intermediate block which was an isobornyl acrylate/isobutyl methacrylate/2-ethylhexyl acrylate random polymer was obtained.

This polymer has a weight-average mass of 77,000 and a number-average mass of 19,000, i.e., a polydispersity index I of 4.05.

### EXAMPLE 3

### Preparation of a Poly(isobornyl Acrylate/Isobornyl Methacrylate/2-Ethylhexyl Acrylate) Polymer

100 g of isododecane were introduced into a 1 liter reactor and the temperature was then increased so as to pass from room temperature (25° C.) to 90° C. over 1 hour. 105 g of isobornyl acrylate, 105 g of isobornyl methacrylate, 110 g of isododecane and 1.8 g of 2,5-bis(2-ethylhexanoylperoxy)-2,5-dimethylhexane (Trigonox® 141 from Akzo Nobel) are then added, at 90° C. and over 1 hour.

The mixture was maintained at 90° C. for 1 hour 30 minutes.

90 g of 2-ethylhexyl acrylate, 90 g of isododecane and 1.2 g of 2,5-bis(2-ethylhexanoylperoxy)-2,5-dimethylhexane were then introduced into the above mixture, still at 90° C. and over 30 minutes.

The mixture was maintained at 90° C. for 3 hours and was then cooled.

A solution containing 50% polymer active material in isododecane was obtained.

A polymer comprising a poly(isobornyl acrylate/isobornyl methacrylate) first block with a Tg of 110° C., a poly-2-ethylhexyl acrylate second block with a Tg of −70° C. and an intermediate block which was an isobornyl acrylate/isobornyl methacrylate/2-ethylhexyl acrylate random polymer was obtained.

This polymer has a weight-average mass of 103,900 and a number-average mass of 21,300, i.e., a polydispersity index I of 4.89.

### EXAMPLE 4

### Preparation of a Poly(isobornyl Methacrylate/Isobutyl Methacrylate/Isobutyl Acrylate) Polymer

100 g of isododecane were introduced into a 1 liter reactor and the temperature was then increased so as to pass from room temperature (25° C.) to 90° C. over 1 hour. 120 g of isobomyl methacrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) were then added, at 90° C. and over 1 hour.

The mixture was maintained at 90° C. for 1 hour 30 minutes.

90 g of isobutyl acrylate, 90 g of isododecane and 1.2 g of 2,5-bis(2-ethylhexanoylperoxy)-2,5-dimethylhexane were then introduced into the above mixture, still at 90° C. and over 30 minutes.

The mixture was maintained at 90° C. for 3 hours and was then cooled.

A solution containing 50% polymer active material in isododecane was obtained.

A polymer comprising a poly(isobornyl methacrylate/isobutyl methacrylate) first block with a Tg of 95° C., a polyisobutyl acrylate second block with a Tg of −20° C. and an intermediate block which was an isobornyl methacrylate/isobutyl methacrylate/isobutyl acrylate random polymer was obtained.

This polymer has a weight-average mass of 100,700 and a number-average mass of 20,800, i.e., a polydispersity index I of 4.85.

### EXAMPLE 5

### Preparation of a Poly(isobornyl Acrylate/Isobornyl Methacrylate/isobutyl Acrylate) Polymer

100 g of isododecane were introduced into a 1 liter reactor and the temperature was then increased so as to pass from room temperature (25° C.) to 90° C. over 1 hour. 105 g of isobornyl acrylate, 105 g of isobornyl methacrylate, 110 g of isododecane and 1.8 g of 2,5-bis(2-ethylhexanoylperoxy)-2,5-dimethylhexane (Trigonox® 141 from Akzo Nobel) were then added, at 90° C. and over 1 hour.

The mixture was maintained at 90° C. for 1 hour 30 minutes.

90 g of isobutyl acrylate, 90 g of isododecane and 1.2 g of 2,5-bis(2-ethylhexanoylperoxy)-2,5-dimethylhexane were then introduced into the above mixture, still at 90° C. and over 30 minutes.

The mixture was maintained at 90° C. for 3 hours and was then cooled.

A solution containing 50% polymer active material in isododecane was obtained.

A polymer comprising a poly(isobornyl acrylate/isobornyl methacrylate) first block with a Tg of 110° C., a polyisobutyl acrylate second block with a Tg of −20° C. and an intermediate block which was an isobornyl acrylate/isobornyl methacrylate/isobutyl acrylate random polymer was obtained.

This polymer has a weight-average mass of 151,000 and a number-average mass of 41,200, i.e., a polydispersity index I of 3.66.

### EXAMPLE 6

### Preparation of a Poly(isobornyl Acrylate/isobutyl Methacrylate/isobutyl Acrylate) Polymer

100 g of isododecane were introduced into a 1 liter reactor and the temperature was then increased so as to pass from room temperature (25° C.) to 90° C. over 1 hour. 120 g of isobornyl acrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) were then added, at 90° C. and over 1 hour.

The mixture was maintained at 90° C. for 1 hour 30 minutes.

90 g of isobutyl acrylate, 90 g of isododecane and 1.2 g of 2,5-bis(2-ethylhexanoylperoxy)-2,5-dimethylhexane were then introduced into the above mixture, still at 90° C. and over 30 minutes.

The mixture was maintained at 90° C. for 3 hours and was then cooled.

A solution containing 50% polymer active material in isododecane was obtained.

A polymer comprising a poly(isobornyl acrylate/isobutyl methacrylate) first block with a Tg of 75° C., a polyisobutyl acrylate second block with a Tg of −20° C. and an intermediate block which was an isobornyl acrylate/isobutyl methacrylate/isobutyl acrylate random polymer was obtained.

This polymer has a weight average mass of 144,200 and a number-average mass of 49,300, i.e., a polydispersity index I of 2.93.

Second Embodiment

According to another aspect of the present disclosure, the block polymer comprises a first block having a glass transition temperature (Tg) of between 20° C. and 40° C., in accordance with the blocks described in c) and a second block having a glass transition temperature of less than or equal to 20° C., as described above in b) or a glass transition temperature of greater than or equal to 40° C., as described in a) above.

For example, the first block with a Tg of between 20° C. and 40° C. is present in an amount ranging from 10% to 85%, such as from 30% to 80%, for instance from 50% to 70% by weight, relative to the total weight of the polymer.

When the second block is a block with a Tg of greater than or equal to 40° C., it can be present, for example, in an amount ranging from 10% to 85% by weight, for instance from 20% to 70%, such as from 30% to 70% by weight, relative to the total weight of the polymer.

When the second block is a block with a Tg of less than or equal to 20° C., it is can be present, for example, in an amount ranging from 10% to 85% by weight, such as from 20% to 70% and from 20% to 50% by weight, relative to the total weight of the polymer.

For example, the first block with a Tg of between 20° C. and 40° C. can be a copolymer derived from monomers, which are such that the corresponding homopolymer has a Tg of greater than or equal to 40° C., and from monomers which are such that the corresponding homopolymer has a Tg of less than or equal to 20° C.

In one embodiment of the present disclosure, the second block with a Tg of less than or equal to 20° C. or with a Tg of greater than or equal to 40° C. is a homopolymer.

Thus, according to a first variant of the second embodiment of the present disclosure, the block polymer comprises:
a first block with a Tg of between 20° C. and 40° C., for example with a Tg ranging from 25° C. to 39° C., which is a copolymer comprising at least one methyl acrylate monomer, at least one methyl methacrylate monomer and at least one acrylic acid monomer,
a second block with a Tg of greater than or equal to 40° C., for example ranging from 85° C. to 125° C., which is a homopolymer composed of methyl methacrylate monomers, and
an intermediate block comprising at least one methyl acrylate, methyl methacrylate monomer, and
an intermediate block comprising methyl methacrylate, at least one acrylic acid monomer and at least one methyl acrylate monomer.

According to a second variant, the block polymer comprises:
a first block with a Tg of between 20° C. and 40° C., for example with a Tg ranging from 21° C. to 39° C., which is a copolymer comprising isobornyl acrylate/isobutyl methacrylate/2-ethylhexyl acrylate,
a second block with a Tg of less than or equal to 20° C., for example ranging from −65° C. to −35° C., which is a methyl methacrylate homopolymer, and
an intermediate block which is an isobornyl acrylate/isobutyl methacrylate/2-ethylhexyl acrylate random copolymer.

According to a third variant, the block polymer comprises:
a first block with a Tg of between 20° C. and 40° C., for example with a Tg ranging from 21° C. to 39° C., which is an isobornyl acrylate/methyl acrylate/acrylic acid copolymer,
a second block with a Tg of greater than or equal to 40° C., for example ranging from 85° C. to 115° C., which is an isobornyl acrylate homopolymer, and
an intermediate block which is an isobornyl acrylate/methyl acrylate/acrylic acid random copolymer.

As a non-limiting illustration, the polymers corresponding to this aspect of the present disclosure have been prepared as follows.

### EXAMPLE 7

### Preparation of a poly(isobornyl Acrylate/isobutyl Methacrylate/2-ethylhexyl Acrylate) Polymer

100 g of isododecane were introduced into a 1 liter reactor and the temperature was then increased so as to pass from room temperature (25° C.) to 90° C. over 1 hour. 54 g of isobornyl acrylate, 75.6 g of isobutyl methacrylate, 50.4 g of 2-ethylhexyl acrylate, 110 g of isododecane and 1.8 g of 2,5-bis(2-ethylhexanoylperoxy)-2,5-dimethylhexane (Trigonox® 141 from Akzo Nobel) were then added, at 90° C. and over 1 hour. This mixture was maintained at 90° C. for 1 hour 30 minutes.

120 g of 2-ethylhexyl acrylate, 90 g of isododecane and 1.2 g of 2,5-bis(2-ethylhexanoylperoxy)-2,5-dimethylhexane were then introduced into the above mixture, still at 90° C. and over 1 hour.

The mixture was maintained at 90° C. for 3 hours and was then diluted and cooled.

A solution containing 50% polymer active material in isododecane was obtained.

The polymer obtained comprises a poly(isobornyl acrylate/isobutyl methacrylate/2-ethylhexyl acrylate) first block with a Tg of 25° C., a poly(2-ethylhexyl acrylate) second block with a Tg of −50° C. and an intermediate block which was an isobornyl acrylate/isobutyl methacrylate/2-ethylhexyl acrylate random polymer.

### EXAMPLE 8

### Preparation of a Poly(methyl Methacrylate/methyl Acrylate/acrylic Acid) Polymer

210 g of ethyl acetate were introduced into a 1 liter reactor and the temperature was then increased so as to pass from room temperature (25° C.) to 78° C. over 1 hour. 54 g of methyl methacrylate, 21 g of acrylic acid, 135 g of methyl acrylate and 1.8 g of 2,5-bis(2-ethylhexanoylperoxy)-2,5-dimethylhexane (Trigonox® 141 from Akzo Nobel) were then added, at 78° C. and over 1 hour.

The mixture was maintained at 90° C. for 1 hour.

90 g of methyl methacrylate, 90 g of ethyl acetate and 1.2 g of 2,5-bis(2-ethylhexanoylperoxy)-2,5-dimethylhexane were then introduced into the above mixture, still at 78° C. and over 1 hour.

The mixture was maintained at 78° C. for 3 hours and was then diluted with 150 g of ethyl acetate and cooled.

A solution containing 40% polymer active material in ethyl acetate was obtained.

The polymer obtained comprises a poly(methyl acrylate/methyl methacrylate/acrylic acid) first block with a Tg of 35° C., a poly(methyl methacrylate) second block with a Tg of 100° C. and an intermediate block which is a methyl methacrylate/acrylic acid/polymethyl acrylate random polymer.

This polymer has a weight-average mass of 141,000 and a number-average mass of 50,000, i.e., a polydispersity index I of 2.82.

For example, the composition according to the present disclosure can be a leave-in composition.

Generally, the first composition of the product of the invention comprises from 0.1% to 60% by weight of active material (or of solids) of block polymer, such as from 0.5% to 50% by weight, and for instance from 1% to 40% by weight, relative to the total weight of the composition.

Second Composition

The cosmetic product according to the present disclosure comprises at least one second composition comprising a second cosmetically acceptable medium.

When the cosmetic product as disclosed herein is a makeup product, the second composition may be any cosmetic makeup composition known to those skilled in the art. For instance, the second composition may be a lipstick, a mascara, a gloss, an eyeshadow, a foundation or a nail varnish. The second composition, for example, can be chosen such that it improves at least one cosmetic property of the first composition when the first composition is applied alone to the said keratin material. For instance, the second composition may improve the comfort of the first composition or reduce its tacky nature.

The second composition may also be chosen such that the product, once applied to the keratin materials, has satisfactory transfer-resistance, gloss and staying-power properties.

For example, the mean gloss measured at 20° of the first composition, once spread onto a support, is greater than or equal to 30, such as greater than or equal to 40, for instance greater than or equal to 50 and greater than or equal to 60 out of 100. Thus, according to one embodiment, the mean gloss of the product measured at 20°, the product being spread onto a support, is greater than or equal to 30, such as greater than or equal to 40, and greater than or equal to 50, for instance greater than or equal to 60 out of 100.

Mean Gloss

As used herein, the term “mean gloss” means the gloss as may be conventionally measured using a glossmeter by the following method:

A coat ranging from 50 μm to 150 μm in thickness of a composition is spread using an automatic spreader onto a Leneta brand contrast card of reference Form 1A Penopac. The composition may be the first or the second composition of the product. The coat covers at least the white background of the card. The deposit is left to dry for 24 hours at a temperature of 30° C., and the gloss at 200 is then measured on the white background using a Byk Gardner brand glossmeter of reference microTri-Gloss.

This measurement, which ranges from 0 to 100, is repeated at least three times, and the mean gloss is the mean of the at least three measurements taken.

When it is desired to measure the gloss of the product according to the present disclosure, a first coat of one of the two compositions is spread onto the contrast card, under the same conditions as described above. This first coat is left to dry, and a second coat of the other composition is then spread on top, under the same conditions as described above.

The mean gloss of the first composition, or of the second composition, or of the product of the present disclosure, measured at 20° can be for example, greater than or equal to 30, such as greater than or equal to 35, greater than or equal to 40, greater than or equal to 45, and for instance, greater than or equal to 50 out of 100, such as greater than or equal to 55, greater than or equal to 60, greater than or equal to 65, for example, greater than or equal to 70 or, for instance, greater than or equal to 75 out of 100. For certain compositions according to the present disclosure, such as nail varnishes, the gloss measured at 20° may be greater than or equal to 70, or even 80 out of 100.

In one aspect of the present disclosure, for example, the mean gloss of the first composition, or of the second composition, or of the product of the invention, once spread onto a support, measured at 60° is greater than or equal to 50, such as greater than or equal to 60, greater than or equal to 65, greater than or equal to 70, and for instance, greater than or equal to 75, greater than or equal to 80, greater than or equal to 85 or, for example, greater than or equal to 90 out of 100.

The measurement of the mean gloss at 60° is performed under the same conditions as those described previously to measure the mean gloss at 20°.

The first composition and/or the second composition may also be chosen such that the product, once applied to the keratin materials, has satisfactory transfer-resistant, gloss and staying-power properties. For example, the first composition can have a transfer index of less than 40, such as less than or equal to 30, less than or equal to 20, less than or equal to 15, less than or equal to 10, less than or equal to 5 and for instance, less than or equal to 2 out of 100.

The second composition may be chosen such that the transfer index of the product of the present disclosure is less than or equal to 40, such as less than or equal to 30, less than or equal to 20, less than or equal to 15, less than or equal to 10 and for instance, less than or equal to 5.

Transfer Index

The transfer index may be measured according to the following method:

A support (rectangle of 40 mm×70 mm and 3 mm thick) of polyethylene foam that is adhesive on one of the faces, having a density of 33 kg/m³ (sold under the name RE40X70EP3 from the company Joint Technique Lyonnais Ind) is preheated on a hotplate maintained at a temperature of 40° C. in order for the surface of the support to be maintained at a temperature of 33° C.±1° C.

When it is desired to measure the transfer index of the first composition, the first composition is applied over the entire non-adhesive surface of the support, by spreading it using a fine brush to obtain a deposit of about 15 μm of the composition, while leaving the support on the hotplate, and the support is then left to dry for 30 minutes.

When it is desired to measure the transfer index of the product of the present disclosure, the first composition is applied over the entire non-adhesive surface of the support, by spreading it using a fine brush to obtain a deposit of about 15 μm of the composition, while leaving the support on the hotplate, and the support is then dried for 30 minutes. The second composition is then applied.

After drying, the support (coated with the first composition or the product) is bonded via its adhesive face onto an anvil of diameter 20 mm and equipped with a screw pitch. The support/deposit assembly is then cut up using a punch 18 mm in diameter. The anvil is then screwed onto a press (Statif Manuel Imada SV-2 from the company Someco) equipped with a tensile testing machine (Imada DPS-20 from the company Someco).

White photocopier paper of 80 g/m² is placed on the bed of the press and the support/deposit assembly is then pressed on the paper at a pressure of 2.5 kg for 30 seconds. After removing the support/deposit assembly, some of the deposit is transferred onto the paper. The color of the deposit transferred onto the paper is then measured using a Minolta CR300 calorimeter, the color being characterized by the L*, a*, b* calorimetric parameters. The colorimetric parameters L*₀, a*₀ and b*₀ of the color of the white paper used is determined.

The difference in color ΔE1 between the color of the deposit transferred relative to the color of the white paper is then determined by means of the following relationship.
ΔE1=\sqrt{(L*−L₀* )²+(a*−a₀*)²+(b*−b₀*)²}

Moreover, a total transfer reference is prepared by applying the composition or the product directly onto a paper identical to the one used previously, at room temperature (25° C.), by spreading the composition using a fine brush and so as to obtain a deposit of about 15 μm of the composition, and the deposit is then left to dry for 30 minutes at room temperature (25° C.). After drying, the calorimetric parameters L*′, a*′ and b*′ of the color of the deposit placed on the paper, corresponding to the reference color of total transfer, is measured directly. The calorimetric parameters L*′₀, a*′₀ and b*′₀ of the color of the white paper used are determined.

The difference in color ΔE2 between the reference color of total transfer relative to the color of the white paper are then determined by means of the following relationship.
ΔE2=\sqrt{(L*′−L₀*′)²+(a*′−a₀*′)²+(b*′−b₀*′)²}

The transfer of the composition or of the product, expressed as a percentage, is equal to the ratio:
100×ΔE1/ΔE2

The measurement is performed on four supports in succession and the transfer value corresponds to the mean of the four measurements obtained with the four supports. The transfer index is equal to the mean of these four measurements.

Organic Liquid Medium

The organic medium of the first composition may comprise any cosmetically acceptable, oil, chosen for example, from carbon-based oils, hydrocarbon-based oils, fluoro oils and/or silicone oils of mineral, plant or synthetic origin, alone or as a mixture provided that they form a uniform and macroscopically stable mixture and that they are compatible with the intended use.

The organic medium of the first composition may be present in an amount ranging from 5% to 90% by weight, relative to the total weight of the composition, such as from 20% to 85% by weight, relative to the total weight of the composition. In one embodiment of the present disclosure, it represents at least 30% of the total weight of the composition.

According to another embodiment, the organic medium comprises at least one volatile oil. These oils may be hydrocarbon-based oils or silicone oils optionally comprising alkyl or alkoxy groups that are pendent or at the end of a silicone chain.

Among the volatile silicone oils that may be used as disclosed herein, non-limiting mention may be made of linear or cyclic silicones with a viscosity at room temperature of less than 8 cSt and for instance, comprising from 2 to 7 silicon atoms, these silicones optionally comprising alkyl or alkoxy groups comprising from 1 to 10 carbon atoms. Further among volatile silicone oils that may be used as disclosed herein, non-limiting mention may be made for example, of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, and mixtures thereof.

Other non-limiting examples of volatile oils that may be used as disclosed herein include C₈-C₁₆ isoalkane oils (also known as isoparaffins), for instance isododecane, isodecane and isohexadecane and, for example, the oils sold under the trade names Isopar and Permethyl, such as isododecane (Permethyl 99A).

The volatile oils can be present in the first composition in an amount ranging from 5% to 85%, such as from 20% to 75% by weight, relative to the total weight of the first composition.

According to one embodiment of the present disclosure, the fatty phase comprises at least one apolar or sparingly polar non-volatile oil.

Among the sparingly polar non-volatile oils that may be used as disclosed herein, non-limiting mention may be made of apolar oils or, for example, oils comprising an alkyl chain for instance ranging from oC₃-C₄₀. Further non-limiting examples of apolar or sparingly polar oils that may be mentioned include:
linear or branched hydrocarbons such as liquid paraffin, liquid petroleum jelly, light naphthalene, hydrogenated polyisobutene;
hydrocarbon-based oils of animal origin, for instance squalene;
fatty alcohols;
hydrocarbon-based plant oils such as liquid triglycerides of fatty acids of at least 10 carbon atoms;
synthetic esters and ethers, such as of fatty acids, for instance the oils of formula R₁CO(O)ₓR₂ wherein R₁ is chosen from acid residues comprising from 2 to 29 carbon atoms with x being equal to 0 or 1 and R₂ is chosen from hydrocarbon-based chains comprising from 3 to 30 carbon atoms, for instance tributyl acetyl citrate, oleyl erucate, 2-octyldodecyl behenate, triisoarachidyl citrate, isocetyl stearoylstearate or octyldodecanyl stearoylstearate, n-propyl acetate, tridecyl trimellitate, diisocetyl dodecanedioleate or stearate, arachidyl propionate, dibutyl phthalate, propylene carbonate or octyldodecyl pentanoate; polyol esters, for instance vitamin F, sorbitan isostearate and glyceryl or diglyceryl triisostearate;
silicone oils such as polydimethylsiloxanes (PDMS), optionally comprising a C₃-C₄₀ alkyl or alkoxy chain or a phenylated chain, such as optionally fluorinated phenyltrimethicones and polyalkylmethylsiloxanes, for instance polymethyltrifluoropropyldimethylsiloxanes, or with functional groups such as hydroxyl, thiol and/or amine groups; polysiloxanes modified with fatty acids, fatty alcohols or polyoxyalkylenes,
fluoro oils; and
mixtures thereof.

The apolar or sparingly polar non-volatile oils may be present in the first composition in an amount ranging from 0.1% to 20% by weight, relative to the total weight of the first composition, such as from 0.5% to 10% and from 1% to 5% by weight, relative to the total weight of the first composition.

For example, the non-volatile oil may be chosen from hydrocarbons, for instance alkanes such as hydrogenated polyisobutene, from phenylsilicone oils, and mixtures thereof.

The phenylsilicone oils that may be used according to the present invention for example, can have a viscosity, measured at 25° C. and atmospheric pressure, ranging from 5 to 100,000 cSt ,and such as from 5 to 10,000 cSt.

The silicone oil may be, for example, a phenyl trimethicone, a phenyl dimethicone, a phenyltrimethylsiloxydiphenylsiloxane, a diphenyl dimethicone, a diphenylmethyldiphenyltrisiloxane or a mixture of various phenylsilicone oils, and may, for instance, correspond to formula (A): 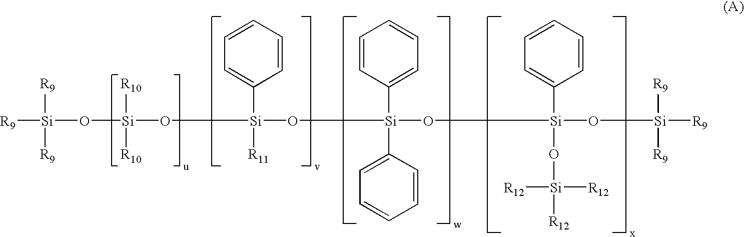
wherein:
R₉ and R₁₂, which may be identical or different, are chosen from C₁-C₃₀ alkyl radicals, aryl radicals and aralkyl radicals,
R₁₀ and R₁₁, which may be identical or different, are chosen from C₁-C₃₀ alkyl radicals and aralkyl radicals,
u, v, w and x are each independently integers ranging from 0 to 900, with the proviso that the sum v+w+x is not 0, and that the sum u+v+w+x ranges from 1 to 900; for example, u+v+w+x can range from 1 to 800.

For example, R₉ can be chosen from C₁-C₂₀ alkyl radicals, phenyl radicals and aralkyl radicals of the type R′-C₆H₅, wherein R′ is chosen from C₁-C₅ alkyl radicals, R₁₀ and R₁₁, which may be identical or different, can be chosen from C₁-C₂₀ alkyl radicals and aralkyl radicals of the type R′-C₆H₅, wherein R′ is chosen from C₁-C₅ alkyl radicals, and R₁₂ is chosen from C₁-C₂₀ alkyl radicals.

As a further example, R₉ can be chosen from methyl, ethyl, propyl, isopropyl, decyl, dodecyl and octadecyl radicals, or alternatively from phenyl, tolyl, benzyl and phenethyl radicals; R₁₀ and R₁₁, which may be identical or different, can be chosen from methyl, ethyl, propyl, isopropyl, decyl, dodecyl and octadecyl radicals, or alternatively from tolyl, benzyl and phenethyl radicals, and R₁₂ can be chosen from methyl, ethyl, propyl, isopropyl, decyl, dodecyl and octadecyl radicals.

As yet another example, the phenylsilicone oil can be an oil of low viscosity of formula (A) with the sum u+v+w+x ranging from 1 to 150, such as from 1 to 100, and from 1 to 50.

For instance, the low-viscosity phenylsilicone oil can be chosen from those of formula (III): 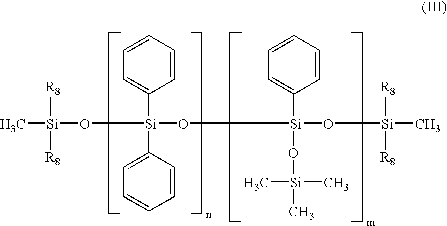
wherein
R₈ is chosen from C₁-C₃₀ alkyl radicals, aryl radicals and aralkyl radicals,
n is an integer ranging from 0 to 100, for instance less than 100,
m is an integer ranging from 0 to 100, with the proviso that the sum m+n ranges from 1 to 100, for instance less than 100.

For example, R₈ can be chosen from C₁-C₂₀ alkyl radicals, phenyl radicals and aralkyl radicals of the type R′-C₆H₅, wherein R′ is chosen from C₁-C₅ alkyl radicals.

As a further example, R₈ can be chosen from methyl, ethyl, propyl, isopropyl, decyl, dodecyl and octadecyl radicals, or alternatively from phenyl, tolyl, benzyl and phenethyl radicals. In one embodiment of the present disclosure, R₈ is a methyl radical.

Among the low-viscosity phenylsilicone oils that may be used in the present disclosure, non-limiting mention may be made of the oils DC556 (22.5 cSt) and SF558 (10-20 cSt) from Dow Corning, the oil Abil AV8853 (4-6 cSt) from Goldschmidt, the oil Silbione 70 633 V 30 (28 cSt) from Rhone-Poulenc, the oils 15 M 40 (50 to 100 cSt) and 15 M 50 (20 to 25 cSt) from PCR, the oils SF 1550 (25 cSt) and PK 20 (20 cSt) from Bayer, the oil Belsil PDM 200 (200 cSt) from Wacker, and the oils KF 53 (175 cSt), KF 54 (400 cSt) and KF 56 (14 cSt) from Shin-Etsu. The values in parentheses represent the viscosities at 25° C.

The organic medium may also comprise at least one polar oil chosen from fatty acid esters comprising from 7 to 29 carbon atoms, for instance diisostearyl malate, isopropyl palmitate, diisopropyl adipate, caprylic/capric acid triglycerides such as those sold by the company Stearineries Dubois or those sold under the names Miglyol 810, 812 and 818 by the company Dynamit Nobel, Shea butter oil, isopropyl myristate, butyl stearate, hexyl laurate, diisopropyl adipate, isononyl isononate, 2-hexyldecyl laurate, 2-octyldecyl palmitate, 2-octyldodecyl myristate or lactate, 2-diethylhexyl succinate, 2-ethylhexyl palmitate, 2-octyldodecyl stearate or castor oil; esters of lanolic acid, of lauric acid or of stearic acid; higher fatty alcohols (ranging from 7 to 29 carbon atoms) such as stearyl alcohol, linoleyl alcohol, linolenyl alcohol, isostearyl alcohol, 2-octyidodecanol, decanol, dodecanol, octadecanol or oleyl alcohol; higher fatty acids (ranging from 7 to 29 carbon atoms) such as myristic acid, palmitic acid, stearic acid, behenic acid, oleic acid, linoleic acid, linolenic acid or isostearic acid; and mixtures thereof.

These non-volatile polar oils may be present in the composition in an amount ranging from 0.1% to 10%, such as from 1% to 5% by weight, relative to the total weight of the composition.

In one embodiment of the present disclosure, when the first or the second composition is intended to be applied to the nails, the cosmetically acceptable medium comprises, for example, at least one solvent chosen from:
ketones that are liquid at room temperature, such as methyl ethyl ketone, methyl isobutyl ketone, diisobutyl ketone, isophorone, cyclohexanone or acetone;
alcohols that are liquid at room temperature, such as ethanol, isopropanol, diacetone alcohol, 2-butoxyethanol or cyclohexanol;
glycols that are liquid at room temperature, such as ethylene glycol, propylene glycol, pentylene glycol or glycerol;
propylene glycol ethers that are liquid at room temperature, such as propylene glycol monomethyl ether, propylene glycol monomethyl ether acetate or dipropylene glycol mono-n-butyl ether;
cyclic ethers such as y-butyrolactone;
short-chain esters (comprising from 3 to 8 carbon atoms in total) such as ethyl acetate, methyl acetate, propyl acetate, isopropyl acetate, n-butyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides such as dimethyl sulfoxide;
aldehydes that are liquid at room temperature, such as benzaldehyde or acetaldehyde;
heterocyclic compounds such as tetrahydrofuran;
propylene carbonate or ethyl 3-ethoxypropionate; and
mixtures thereof.

In another embodiment of the present disclosure, when the first or the second composition is intended to be applied to the nails, the cosmetically acceptable medium comprises, for example, at least one solvent chosen from methyl acetate, isopropyl acetate, methoxypropyl acetate, butyl lactate, acetone, methyl ethyl ketone, diacetone alcohol, y-butyrolactone, tetrahydrofuran, propylene carbonate, ethyl 3-ethoxypropionate, dimethyl sulfoxide, and mixtures thereof.

The first composition may comprise, besides the block polymer, an additional polymer such as a film-forming polymer. The second composition may also contain a film-forming polymer. According to the present disclosure, the term “film-forming polymer” means a polymer capable of forming, by itself or in the presence of an auxiliary film-forming agent, a continuous film that adheres to a support, such as to keratin materials.

Among the film-forming polymers that may be used in the composition of the present disclosure, non-limiting mention may be made of synthetic polymers, of free-radical type or of polycondensate type, polymers of natural origin, and mixtures thereof.

The film-forming polymer may be chosen, for example, from cellulose-based polymers such as nitrocellulose, cellulose acetate, cellulose acetobutyrate, cellulose acetopropionate or ethylcellulose, or from polyurethanes, acrylic polymers, vinyl polymers, polyvinyl butyrals, alkyd resins, resins derived from aldehyde condensation products, such as arylsulfonamide-formaldehyde resins, for instance toluenesulfonamide-formaldehyde resin, and arylsulfonamide epoxy resins.

Further non-limiting examples of film-forming polymers that may be used as disclosed herein include nitrocellulose RS ⅛ sec.; RS ¼ sec.; ½ sec.; RS 5 sec.; RS 35 sec.; RS 75 sec.; RS 150 sec.; AS ¼ sec.; AS ½ sec.; SS ¼ sec.; SS ½ sec.; SS 5 sec., sold for example, by the company Hercules; the toluenesulfonamide-formaldehyde resins “Ketjentflex MS80” from the company Akzo or “Santolite MHP” and “Santolite MS80” from the company Faconnier or “Resimpol 80” from the company Pan Americana, the alkyd resin “Beckosol Ode 230-70-E” from the company Dainippon, the acrylic resin “Acryloid B66” from the company Rohm & Haas, and the polyurethane resin “Trixene PR 4127” from the company Baxenden.

The film-forming polymer may be present in the composition according to the present disclosure in an amount ranging from 0.1% to 60% by weight, such as ranging from 2% to 40% by weight, for instance from 5% to 25% by weight, relative to the total weight of the composition.

The first or the second composition may also comprise at least one plasticizer. The amount of plasticizer may be chosen by a person skilled in the art on the basis of her general knowledge, so as to obtain a composition with cosmetically acceptable properties.

The first composition according to the present disclosure may also comprise, depending on the intended type of application, at least one wax.

For the purposes of the present disclosure, a wax is a lipophilic fatty compound, that is solid at room temperature (25° C.), with a reversible solid/liquid change of state, having a melting point of greater than 40° C. which may be up to 200° C., and having an anisotropic crystal organization in the solid state.

In one aspect of the present disclosure, for example, linear hydrocarbon-based waxes may be used. The melting point of the linear hydrocarbon based waxes may be, for instance, greater than 35° C., for example greater than 55° C., such as greater than 80° C.

Non-limiting examples of the linear hydrocarbon-based waxes that may be used include those chosen from substituted linear alkanes, unsubstituted linear alkanes, unsubstituted linear alkenes and substituted linear alkenes, an unsubstituted compound being a compound of carbon and hydrogen exclusively. The substituents mentioned above do not contain any carbon atoms.

The linear hydrocarbon-based waxes include ethylene polymers and copolymers with a molecular weight ranging from 400 to 800, for example Polywax 500 or Polywax 400 sold by New Phase Technologies. The linear hydrocarbon-based waxes include linear paraffin waxes, for instance the paraffins S&P 206, S&P 173 and S&P 434 from Strahl & Pitsch. The linear hydrocarbon-based waxes include long-chain linear alcohols, for instance products comprising a mixture of polyethylene and of alcohols comprising 20 to 50 carbon atoms, for instance Performacol 425 or Performacol 550 (mixture in 20/80 proportions) sold by New Phase Technologies.

The waxes may be present in the first composition in an amount ranging from 0.5-30%, such as from 5% to 20% and from 5% to 15% by weight, relative to the total weight of the first composition, so as not to excessively reduce the gloss of the composition and of the film applied to the lips and/or the skin.

The cosmetically acceptable medium of the second composition can comprise, for example, a liquid phase that is non-volatile at room temperature and atmospheric pressure. The term “non-volatile liquid phase” means any medium capable of remaining on the skin or the lips for several hours. A non-volatile liquid phase, for instance, has a non-zero vapor pressure at room temperature and atmospheric pressure, of less than 0.02 mmHg (2.66 Pa) such as less than 10⁻³ mmHg (0.13 Pa).

The non-volatile liquid phase of the second composition may be at least one hydrocarbon-based oil, a liquid silicone phase, a fluoro phase that is liquid at room temperature, or a mixture thereof. The term “hydrocarbon-based oil” means oils mainly comprising carbon atoms and hydrogen atoms, such as alkyl or alkenyl chains, for instance alkanes or alkenes, but also oils comprising an alkyl or alkenyl chain comprising one or more ether, ester or carboxylic acid groups.

The non-volatile liquid phase of the second composition can be present in an amount ranging from 1% to 100%, such as from 5% to 95%, from 20% to 80% and for instance from 40% to 80% by weight, relative to the total weight of the second composition.

In one embodiment of the present disclosure, the non-volatile liquid phase of the second composition comprises, for example, a silicone oil, for example a phenylsilicone oil or a polydimethylsiloxane oil.

The non-volatile silicone oil may be chosen from polyalkylsiloxanes, polyarylsiloxanes, polyalkylarylsiloxanes, and mixtures thereof.

The non-volatile silicone oil may be chosen from:
linear and branched non-volatile polydimethylsiloxanes (PDMS);
polydimethylsiloxanes comprising alkyl, alkoxy and phenyl groups, which are pendent or at the end of a silicone chain, these groups comprising from 2 to 24 carbon atoms; and
phenylsilicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

The polyalkylsiloxanes according to the present disclosure include polydimethylsiloxanes, (polydimethylsiloxane) (methylvinylsiloxane) copolymers, poly(dimethylsiloxane)(diphenyl)siloxanes, poly(dimethylsiloxane)(diphenyl)(methylvinylsiloxane) copolymers, and mixtures thereof.

In another embodiment of the present disclosure, the non-volatile silicone oil is chosen from the silicones of formula (II): 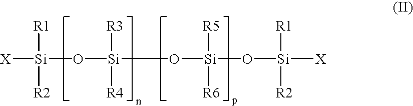
wherein:

R₁, R₂, R₅ and R₆, which may be identical or different, are chosen from alkyl radicals comprising from 1 to 6 carbon atoms,

R₃ and R₄, which may be identical or different, are chosen from alkyl radicals comprising from 1 to 6 carbon atoms and aryl radicals,

X is chosen from alkyl radicals comprising from 1 to 6 carbon atoms, hydroxyl radicals and vinyl radicals,

n and p are chosen so as to give the oil a weight-average molecular mass of less than 200,000 g/mol, such as less than 150,000 g/mol, for instance less than 100,000 g/mol.

Non-volatile silicones of formula (II) include polydimethylsiloxanes with a viscosity ranging from 0.5 to 60,000 cSt, such as from 0.5 to 10,000 cSt, for instance from 0.5 and 1,000 cSt measured according to ASTM standard D-445, for example DC 200 of viscosity 350 cSt sold by Dow Corning.

According to one embodiment of the present disclosure, the second composition comprises a non-volatile silicone of formula (II) such as a polydimethylsiloxane with a viscosity ranging, for example, from 0.5 to 500 cSt, such as from 1 to 10 cSt, for example the polydimethylsiloxane sold under the name DC 200 of viscosity 5 cSt and of molecular weight 800, sold by Dow Corning.

The weight-average molecular mass of the silicone oil may range, for example, from 400 to 200,000, such as from 4,000 to 100,000, from 4,000 and 20,000, for instance, from 400 to 2,000 and from 400 to 1,000 g/mol.

According to one embodiment of the present disclosure, the second composition comprises, for example, a mixture of a polydimethylsiloxane with a molecular mass ranging from 200,000 to 300,000 g/mol and a polydimethylsiloxane with a molecular mass ranging from 400 to 1,000 g/mol.

The weight ratio of the silicone compound of high molecular weight to the liquid silicone compound can range, for example, from 20/80 to 60/40, such as from 35/65 to 45/55.

For example, the non-volatile liquid phase of the second composition can comprise at least one fluoro oil of formula (III): 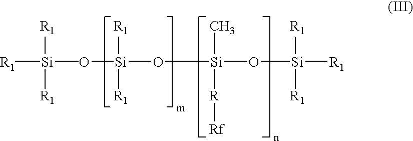
wherein:
R is chosen from linear and branched divalent alkyl groups comprising from 1 to 6 carbon atoms, such as a divalent methyl, ethyl, propyl or butyl group,
Rf is chosen from fluoroalkyl radicals, such as perfluoroalkyl radicals, comprising from 1 to 9 carbon atoms, such as from 1 to 4 carbon atoms,
R₁, which may be identical or different, are chosen from C₁-C₂₀, such as C₁-C₄, alkyl radicals, hydroxyl radicals and phenyl radicals,
m ranges from 0 to 150, such as from 20 to 100, and
n ranges from 1 to 300, such as from 1 to 100.

Non-limiting mention may be made of the oils of formula (III) wherein R1 is methyl, R is ethyl and Rf is CF₃. Further non-limiting mention may be made of fluorosilicone compounds of formula (III) such as those sold by the company Shin Etsu under the names “X22-819”, “X22-820”, “X22-821” and “X22-822” or “FL-100”.

Among the fluoro oils that may also be used as disclosed herein, non-limiting mention may be made of fluoro polyethers chosen from the compounds of formula (IV) below:
R₆—(CF₂—CFR₃—CF₂O)p—(CFR₄—CF₂-O)q-(CFR₅—O)r—R₇ (IV)
wherein:
R₃ to R₆, which may be identical or different, are chosen from monovalent radicals chosen from —F, —(CF₂)n-CF₃ and —O—(CF₂)n-CF₃,
R₇ is chosen from monovalent radicals chosen from —F and —(CF₂)n-CF₃,
with n ranging from 0 to 4 inclusive,
p ranges from 0 to 600, q ranges from 0 to 860, r ranges from 0 to 1500, and p, q and r are integers chosen such that the weight-average molecular mass of the compound ranges from 500 to 100,000, such as from 500 to 10,000.

The fluoro oils may also be chosen from fluoroalkanes chosen from C₂-C₅₀, such as C₅-C₃₀ perfluoroalkanes and fluoroalkanes, such as perfluorodecalin, perfluoroadamantane, bromoperfluorooctyl, and mixtures thereof.

The second composition may optionally comprise a volatile oil. These oils may be hydrocarbon-based oils or silicone oils optionally comprising alkyl or alkoxy groups that are pendent or at the end of a silicone chain, or a mixture of these oils.

For example, the volatile oils can be cosmetic oils chosen from oils with a flash point ranging from 40° C. to 100° C., and mixtures thereof. In addition, the oils may have, for example, a boiling point at atmospheric pressure of less than 220° C., such as less than 210° C., for instance ranging from 110° C. to 210° C. In one embodiment, these volatile oils are not monoalcohols comprising at least 7 carbon atoms.

Among the volatile oils that may be used as disclosed herein, non-limiting mention may be made of linear and cyclic silicone oils with a viscosity at room temperature of less than 8 cSt, and for example, comprising from 2 to 7 silicon atoms, these silicones optionally comprising alkyl or alkoxy groups ranging from 1 to 10 carbon atoms. Among volatile silicone oils that may be used in according to the present disclosure, further non-limiting mention may be made for example, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

Among other volatile oils that may be used as disclosed herein, non-limiting mention may be made of hydrocarbon-based volatile oils comprising from 8 to 16 carbon atoms and mixtures thereof, for instance, C₈-C₁₆ branched alkanes, for instance C₈-C₁₆ isoalkanes (also known as isoparaffins), isododecane, isodecane and isohexadecane, and for example the oils sold under the trade names Isopar or Permethyl, and C₈-C₁₆ branched esters, for instance isohexyl neopentanoate, and mixtures thereof. For example, isododecane (Permethyl 99 A), C₈-C₁₆ isoparaffins, for instance Isopar L, E, G or H, and mixtures thereof, optionally combined with decamethyltetrasiloxane or with cyclopentasiloxane, may also be used.

In general, the amount of volatile oil used is an amount that is sufficient to improve the spreading qualities of the second composition. This amount will be adapted by a person skilled in the art as a function of the intensity of the desired properties.

Moreover, the amount of volatile oil is chosen so that it does not reduce the gloss of the second composition. According to one embodiment, the second composition does not comprise volatile oil.

The second composition, for example, can comprise a high molecular weight polymer that is different from the non-volatile silicone oil described above. When the second composition according to the present disclosure is liquid, it can comprise from 20% to 50% by weight of a high molecular weight polymer. When the second composition according to the present disclosure is solid, for example, it can comprise from 2% to 40% by weight of a high molecular weight polymer.

In one embodiment of the present disclosure, the polymer is a silicone polymer.

The high molecular weight polymer may be liquid or solid at room temperature and its weight-average molecular mass is greater than or equal to 200,000 g/mol, such as from 200,000 to 2,500,000 and from 200,000 to 2,000,000 g/mol. The viscosity of this polymer can range from 10,000 to 5,000,000 cSt, such as from 100,000 to 1,000,000 cSt, and for instance from 300,000 to 700,000 cSt, measured according to ASTM standard D-445.

The high molecular weight polymer may be, for example, an ungrafted polymer, i.e., a polymer obtained by polymerizing at least one monomer, without subsequent reaction of the side chains with another chemical compound. For instance, the polymer can be chosen from dimethiconols, fluorosilicones, dimethicones and mixtures thereof. In one embodiment of the present disclosure, the polymer is a homopolymer.

For further example, the high molecular weight polymer that may be used is a high molecular weight polymer chosen from those of formula (V): 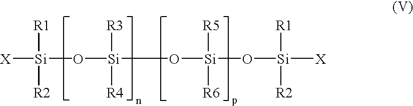
wherein:

R₁, R₂, R₅ and R₆, owhich can be identical or different, are chosen from alkyl radicals comprising from 1 to 6 carbon atoms, optionally substituted with at least one fluorine atom,

R₃ and R₄, which may be identical or different, are chosen from alkyl radicals comprising from 1 to 6 carbon atoms and aryl radicals,

X is chosen from alkyl radicals comprising from 1 to 6 carbon atoms, hydroxyl radicals, vinyl and allyl radicals and alkoxy radicals comprising from 1 to 6 carbon atoms,

n and p are chosen such that the silicone compound has a weight-average molecular mass of greater than or equal to 200,000 g/mol.

In one embodiment, for example, p is equal to 0.

The polymers of formula (V), wherein R₁ to R₆ are methyl groups and X is a hydroxyl group, are dimethiconols. Non-limiting examples that may be mentioned include the polymers of formula (V) such that p is equal to 0 and n ranges from 2,000 to 40,000, such as from 3,000 to 30,000. Non-limiting mention may also be made of polymers with a molecular mass ranging from 1,500,000 to 2,000,000 g/mol.

According to one embodiment of the present disclosure, the high molecular weight polymer is the dimethiconol sold by Dow Corning in a polydimethylsiloxane (5 cSt) under the reference D2-9085, the viscosity of the mixture being equal to 1550 cSt, or the dimethiconol sold by Dow Corning in a polydimethylsiloxane (5 cSt) under the reference DC 1503. In another embodiment, the dimethiconol (of molecular weight equal to 1,770,000 g/mol) sold by Dow Corning under the reference Q2-1403 or Q2-1401, the viscosity of the mixture being equal to 4000 cSt, is used.

Among the high molecular weight polymers that may be used according to the present disclosure, non-limiting mention may be made of those for which:
the substituents R₁ to R₆ and X are methyl groups, for instance the product sold under the name SE30 by the company General Electric, and the product sold under the name AK 500 000 by the company Wacker,
the substituents R₁ to R₆ and X are methyl groups and p and n are such that the molecular weight is 250,000 g/mol, for instance the product sold under the name Silbione 70047 V by the company Rhodia,
the substituents R₁ to R₆ are methyl groups and the substituent X is a hydroxyl group, for instance the products sold under the name Q2-1401 or Q2-1403 by the company Dow Corning,
the substituents R₁, R₂, R₅, R₆ and X are methyl groups, the substituents R₃ and R₄ are aryl groups and n and p are such that the molecular weight of the polymer is 600,000 g/mol, for instance the product sold under the name 761 by the company Rhône-Poulenc.

For example, the high molecular weight silicone polymer may be introduced into the composition in the form of a mixture with a liquid silicone, the viscosity of the liquid silicone ranging from 0.5 to 10,000 cSt, such as from 0.5 to 500 cSt, and for instance, from 1 to 10 cSt. The fluid silicone may be chosen from polyalkylsiloxanes, polyarylsiloxanes, polyalkylarylsiloxanes and mixtures thereof. The liquid silicone may be a volatile silicone such as a cyclic polydimethylsiloxane comprising from 3 to 7—(CH₃)₂SiO— units. The liquid silicone may also be a non-volatile polydimethylsiloxane silicone, for example, with a viscosity ranging from 0.5 to 10,000 cSt, such as a viscosity of 5 cSt, for example the silicone sold under the reference DC 200 by Dow Corning.

The ratio of the high molecular weight silicone polymer in the high molecular weight silicone polymer to the liquid silicone mixture can range, for example from 10/90 to 20/80. The viscosity of the high molecular weight silicone polymer and liquid silicone mixture can range, for instance, from 1,000 to 10,000 cSt.

The high molecular weight dimethicones according to the present disclosure include the dimethicones described in U.S. Pat. No. 4,152,416. They are sold, for example, under the references SE30, SE33, SE54 and SE76. The dimethicones as disclosed herein are, for example, compounds of formula (III) such that R₁ to R₆ and X are methyl radicals and p is equal to zero. The molecular weight of these polymers may range, for example, from 200,000 to 300,000, such as from 240,000 to 260,000 g/mol. Non-limiting examples of the dimethicones according to the present disclosure include polydimethylsiloxanes, (polydimethylsiloxane) (methylvinylsiloxane) copolymers, poly(dimethylsiloxane)(diphenyl)(methylvinylsiloxane) copolymers, and mixtures thereof.

The high molecular weight fluorosilicones according to the present disclosure may have, for example, a molecular weight ranging from 200,000 to 300,000, such as 240,000 to 260,000 g/mol.

According to one aspect of the present disclosure, the second composition as disclosed herein comprises at least one apolar or sparingly polar compound, which may be chosen from oils, gums and/or waxes. The second composition may comprises, for example, more than 70%, such as more than 80% by weight and for instance, 100% by weight of apolar or sparingly polar compounds. These apolar or sparingly polar compounds comprise coloring agents or gelling agents.

According to one embodiment of the present disclosure, the second composition comprises a mixture of a polymer with a weight-average molecular mass of greater than or equal to 200,000 g/mol and a silicone oil as described above.

According to another embodiment of the present disclosure, the second composition is transparent. The term “transparent composition” means a transparent to translucent composition, i.e., a composition which is such that it transmits at least 40% of light, such as at least 50% of light with a wavelength of 750 nm. The transmission is measured using a Cary 300 Scan UV-visible spectrophotometer from the company Varian, according to the following protocol:

The composition is poured above its melting point into a spectrophotometer cuvette of square cross section with a side length of 10 mm. The sample of the composition is then cooled for 24 hours at 35° C., and then kept in a chamber thermostatically maintained at 20° C. for 24 hours.

The light transmitted through the sample of the composition is then measured by spectrophotometer by scanning wavelengths ranging from 700 nm to 800 nm, the measurement being performed in transmission mode. The percentage of light transmitted through the sample of the composition at a wavelength of 750 nm is then determined.

When the second composition is transparent, it may, for example, comprise less than 5%, such as less than 2% and for instance, less than 1% of pigments.

The high molecular weight polymer and liquid silicone compound mixture can be present in the second composition in an amount of more than 70%, such as more than 80% by weight, more than 90% by weight and for instance, more than 95% by weight, relative to the total weight of the second composition.

The second composition may comprise other compounds, which may be, for example, apolar or sparingly polar. These apolar or sparingly polar compounds may be, for instance, silicone compounds, coloring agents or gelling agents.

According to one embodiment of the present disclosure, the second composition comprises only apolar or sparingly polar ingredients.

In another embodiment, the second composition comprises at least one wax, for instance, when the second composition is in solid form.

The waxes may be present in an amount ranging from 0.5% to 30% by weight in the composition, such as from 5% to 20% and for instance, from 5% to 15% of the composition.

In one embodiment, linear hydrocarbon-based waxes are used, wherein their melting point is for instance, greater than 35° C., for example greater than 55° C., such as greater than 80° C. The linear hydrocarbon-based waxes may be chosen from, for example, substituted linear alkanes, unsubstituted linear alkanes, unsubstituted linear alkenes and substituted linear alkenes, an unsubstituted compound comprising exclusively carbon and hydrogen. The substituents mentioned above do not contain carbon atoms.

The linear hydrocarbon-based waxes include ethylene polymers and copolymers with a molecular weight ranging from 400 to 800, for example Polywax 500 or Polywax 400 sold by New Phase Technologies. The linear hydrocarbon-based waxes also include linear paraffin waxes, for instance the paraffins S&P 206, S&P 173 and S&P 434 from Strahl & Pitsch. Further, the linear hydrocarbon-based waxes include long-chain linear alcohols, for instance products comprising a mixture of polyethylene and of alcohols comprising from 20 to 50 carbon atoms, such as Performacol 425 or Performacol 550 (mixture in 20/80 proportions) sold by New Phase Technologies.

The second composition may comprise, for example, a silicone wax, such as a dimethicone comprising alkyl groups at the end of a chain. These alkyl groups may comprise, for instance, more than 18 carbon atoms, such as from 20 to 50 and from 30 to 45 carbon atoms.

The silicone wax, for example, can be chosen from those of formulae (VI) and (VII): 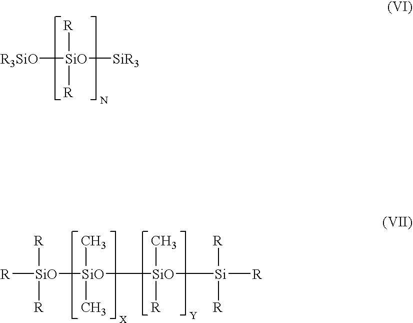
wherein R is an alkyl group, X is greater than or equal to zero and N and Y are greater than or equal to one.

R comprises from 1 to 50 carbon atoms when the compound of formulae (VI) or (VII) is solid at room temperature.

Non-limiting examples of silicone waxes include:
C₂₀₋₂₄ alkyl methicone, C₂₄₋₂₈ alkyl dimethicone, C₂₀₋₂₄ alkyl dimethicone and C₂₄₋₂₈ alkyl dimethicone sold by Archimica Fine Chemicals under the reference Silcare 41M40, SilCare 41M50, SilCare 41M70 and SilCare 41M80,
the stearyl dimethicones of reference SilCare 41M65 sold by Archimica or of reference DC-2503 sold by Dow Corning,
the stearoxytrimethylsilanes sold under the reference SilCare 1 M71 or DC-580,
the products Abil Wax 9810, 9800 or 2440 from Wacker-Chemie GmbH,
the C₃₀₋₄₅ alkyl methicones sold by Dow Corning under the reference AMS-C30 Wax, and also the C₃₀₄₅ alkyldimethicones sold by General Electric under the reference SF 1642 or SF 1632.

The first and/or the second composition of the cosmetic product according to the present disclosure can further comprise a coloring agent that may be chosen from water-soluble and liposoluble dyes, pigments and nacres, and mixtures thereof.

The term “pigments” should be understood as meaning white or colored, mineral or organic particles, which are insoluble in the liquid organic phase and which are intended to color and/or opacify the first composition.

The term “nacres” should be understood as meaning iridescent particles produced for example, by certain molluscs in their shell, or else synthesized, which are insoluble in the medium of the first composition.

The term “dyes” should be understood as meaning generally organic compounds that are soluble in fatty substances, for instance oils, or soluble in an aqueous-alcoholic phase.

The liposoluble dyes include, as a non-limiting example, Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The water-soluble dyes may include, for example, beetroot juice, methylene blue and caramel.

The pigments may be interference or non-interference, mineral and/or organic, white and colored pigments. Among the mineral pigments that may be used, non-limiting mention may be made of titanium dioxide, optionally surface-treated, zirconium oxide or cerium oxide, and also zinc oxide, iron oxide (black, yellow, brown and red) and chromium oxide, manganese violet, ultramarine blue, chromium hydrate and ferric blue. Among the organic pigments that may be used, non-limiting mention may be made of are carbon black, pigments of barium, strontium, calcium and aluminium organic lake type, including those submitted for certification by the Food and Drug Administration (FDA) (for example D&C or FD&C) and those exempt from FDA certification, for instance lakes based on cochineal carmine.

The nacres and nacreous pigments may be chosen from white nacreous pigments such as mica coated with titanium or with bismuth oxychloride, colored nacreous pigments such as titanium mica with iron oxides, titanium mica with, for instance, ferric blue or chromium oxide, titanium mica with an organic pigment of the abovementioned type, and also nacreous pigments based on bismuth oxychloride. Pigments with goniochromatic properties and/or pigments with a metallic effect as described in the French patent application No. FR 0 209,246, the content of which is incorporated by reference into the present patent application, may thus be used.

In general, the coloring agents can be present in an amount ranging from 0.001% to 60%, such as from 0.01% to 50%, for instance from 0.1% to 40% by weight, relative to the total weight of each first and second composition.

The first and/or the second composition of the present disclosure may also comprise at least one cosmetic or dermatological active agent such as those conventionally used. Among the cosmetic, dermatological, hygiene and/or pharmaceutical active agents that may be used in the composition of the present disclosure, non-limiting mention may be made of moisturizers, vitamins, essential fatty acids, sphingolipids and sunscreens. These active agents are used in the usual amount for those skilled in the art, for instance, in an amount ranging from 0 to 20%, such as from 0.001% to 15%, relative to the total weight of the first or second composition.

The composition may also comprise any other additive usually used in such compositions, such as water, antioxidants, fragrances, preserving agents and essential oils.

Needless to say, a person skilled in the art will take care to select this or these optional compounds, and/or the amount thereof, such that the beneficial properties of the compositions and final product are not, or are not substantially, adversely affected by the envisaged addition.

The compositions of the product may be in the form of a cast product, for example in the form of a stick or wand, or in the form of a dish that may be used by direct contact or with a sponge. For example, the compositions of the product can be in the form of cast foundations, cast makeup rouges, eyeshadows, lipsticks, lipcare bases, lipcare balms, concealer products and nail varnishes. They may also be in the form of a soft paste or a gel, a more or less fluid cream, or a liquid packaged in a tube.

The compositions of the product according to the present disclosure may comprise, for example, a cosmetic care composition for the face, the neck, the hands or the body (for example a care cream, an antisun oil or a body gel), a makeup composition (for example a makeup gel, a cream or a stick) or a composition for artificially tanning or protecting the skin. The compositions of the product according to the present disclosure may also be in the form of a care composition for the skin and/or the integuments or in the form of an antisun composition or a body hygiene composition, such as in the form of a deodorant. In this case, they may be for example, in uncolored form. They may then be used as a care base for the skin, the integuments or the lips (lip balms, for protecting the lips against the cold and/or sunlight and/or the wind, or care creams for the skin, the nails or the hair).

For the purposes of the present disclosure, the term “cosmetically acceptable” means a composition of pleasant appearance, odor and feel.

Each composition of the product according to the present disclosure may be in any galenical form normally used for topical application, for instance, in the form of an oily or aqueous solution, an oily or aqueous gel, an oil-in-water or water-in-oil emulsion, a multiple emulsion, a dispersion of oil in water using vesicles, the vesicles being located at the oil/water interface, or a powder. Each composition may be fluid or solid.

According to one embodiment of the present disclosure, the at least one first and/or the at least one second composition, have a continuous fatty phase for example, in anhydrous form and may comprise less than 5% water, such as less than 1% water, relative to the total weight of the first or second composition.

Each of the first and second compositions may have the appearance of a lotion, a cream, an ointment, a soft paste, a salve, a solid that has been cast or molded, such as a stick or a dish, or a compacted solid.

Each composition may be packaged separately in the same packaging article, for example in a two-compartment pen, the base composition being delivered from one end of the pen and the top composition being delivered from the other end of the pen, each end being especially closed in a leaktight manner with a lid.

Alternatively, each of the compositions may be packaged in a different packaging article.

For example, the composition that is applied as the first coat may be in liquid or pasty form, which is highly desirable in the case of a lipstick or an eyeliner.

The product according to the present disclosure may be for example, used for making up the skin and/or the lips and/or the integuments depending on the nature of the ingredients used. For instance, the product of the present disclosure may be in the form of a solid foundation, a lipstick wand or paste, a concealer product, an eye contour product, an eyeliner, a mascara, an eyeshadow, a body-makeup product or a skin-coloring product.

For example, the second composition has care, gloss or transparency properties.

Another aspect of the present disclosure is a lip product, a varnish, a mascara, a foundation, a tattoo, a makeup rouge or an eyeshadow comprising at least one first and at least one second composition as described above.

The compositions of the product of the present disclosure may be obtained by heating the various constituents to the melting point of the highest-melting waxes, followed by casting the molten mixture in a mold (dish or finger stall). They may also be obtained by extrusion, as described in European patent application EP-A-0 667 146.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples below serve to illustrate the present disclosure in a non-limiting manner. The percentages are percentages by weight.

### Examples 9 to 13

### First Composition

| | Example | | | | |
|---|---|---|---|---|---|
| | 9 | 10 | 11 | 12 | 13 |
| Polymer of Example 2 | 90.7 | | | | |
| Polymer of Example 3 | | 90.7 | | | |
| Polymer of Example 6 | | | 90.7 | | |
| Polymer of Example 5 | | | | 90.7 | |
| Polymer of Example 4 | | | | | 90.7 |
| Hydrogenated polyisobutene | 2.1 | 2.1 | 2.1 | 2.1 | 2.1 |
| Octyldodecanol | 0.9 | 0.9 | 0.9 | 0.9 | 0.9 |
| Phenyl trimethicone | 2.1 | 2.1 | 2.1 | 2.1 | 2.1 |
| (DC 556, 20 cSt, Dow Corning) | | | | | |
| Copolymer vinylpyrrolidone/ | 1.2 | 1.2 | 1.2 | 1.2 | 1.2 |
| 1-eicosene | | | | | |
| (Antaron V-220, ISP) | | | | | |
| Pigments | 3 | 3 | 3 | 3 | 3 |

Procedure
1. A ground pigmentary mixture of the pigments in the oily phase was prepared by treating the mixture three times in a three-roll mill.
2. The ground material required for the composition and the other ingredients were weighed out in a beaker.
3. The mixture was stirred using a Rayneri blender for 45 minutes.
4. The formula was cast in isododecane-leaktight cooling boxes.
   Gloss Measurement
1. Films with a wet thickness of 50 and/or 150 μm were prepared using a mechanical applicator. The depositions were made on a contrast card.
2. The films were left to dry for 24 hours at a regulated temperature of 30° C.
3. The gloss measurements were performed using a Byk Gardner micro-tri-gloss glossmeter with measuring angles of 20° and 60°.

The gloss results obtained are given in the table below:

Wet 150 μm deposit

| | | | Mean | Standard |
|---|---|---|---|---|
| Angle | Deposit | Example | (%) | deviation |
| Angle of | 150 μm | Example 9 | 54.2 | 2.0 |
| 20° | deposit | Example 10 | 41.0 | 3.5 |
| Angle of | 150 μm | Example 9 | 75.7 | 0.8 |
| 60° | deposit | Example 10 | 73.6 | 1.6 |

Wet 50 μm deposit (closer when dry to the thickness applied to the lips)

| | | | Mean | Standard |
|---|---|---|---|---|
| Angle | Deposit | Example | (%) | deviation |
| Angle of | 50 μm | Example 9 | 47.6 | 1.2 |
| 20° | deposit | Example 10 | 42.6 | 5.2 |
| Angle of | 50 μm | Example 9 | 69.3 | 0.7 |
| 60° | deposit | Example 10 | 74.8 | 1.0 |

### EXAMPLE 14

### Second Liquid Composition

| | |
|---|---|
| Polydimethylsiloxane sold under the reference Silbione 70047 V by | 40% |
| Rhodia | |
| (500 000 cSt-250 000 g/mol) | |
| Polydimethylsiloxane sold by the company Dow Corning under the | 60% |
| reference DC200 (5 cSt) | |

The two ingredients were mixed together at 70° C. using a Rayneri blender.

### EXAMPLE 15

### Second Solid Composition

| | % by weight |
|---|---|
| Silicone oil (PDMS) | 25 |
| DC200 from Dow Corning (5 cSt) | |
| Dimethicone (and) Dimethiconol | 61 |
| D2-9085 from Dow Corning (1550 cSt) | |
| Trifluoropropyl dimethicone (100 cSt) | 1 |
| X22-819 from Shin-Etsu | |
| C30-45 Alkyl dimethicone | 5 |
| (SF 1642 from GE Bayer Silicone) | |
| Polyethylene wax (weight-average MW 500) | 8 |

The silicone oil, the dimethiconol and the fluoro dimethicone were mixed together while hot until a uniform mixture was formed. The C₃₀-C₄₅ alkyl dimethicone was then added to the above mixture brought to 110° C. The polyethylene wax was then added portionwise until a uniform mixture was obtained. The mixture was cooled to 90-95° C. and then poured into the molds, which were placed at −20° C. for 30 minutes. Finally, the sticks were stripped from the molds.

### EXAMPLE 16

### Measurement of the Transfer Resistance of the Product Comprising the Composition of Example 11 as first composition and the composition of Example 15 as Second Composition

The transfer index of the product comprising the composition of Example 11 as first composition and the composition of Example 15 as second composition was measured according to the method described above.

The transfer index of the comparative example: product Lipfinity (shade 70), comprising a liquid lipstick and a colorless balm, was measured by proceeding as previously.

| Product whose transfer resistance is | Transfer value (in |
|---|---|
| evaluated | %) |
| Composition of Example 11 onto which is | 4 ± 0.5 |
| applied the composition of Example 15 | |
| Lipfinity product from Max Factor | 5 ± 0 |

### EXAMPLE 17

### Nail Varnish Kit

| First composition | | |
|---|---|---|
| Polymer of Example 1 | 23.8 | g AM |
| Butyl acetate | 24.99 | g |
| Isopropanol | 10.71 | g |
| Hexylene glycol | 2.5 | g |
| DC Red 7 Lake | 1 g | |
| Hectorite modified with | 1.3 | g |
| distearyldimethylbenzylammonium chloride (Bentone ® | | |
| 27V from Elementis) | | |
| Ethyl acetate | qs 100 | g |
| Second composition | | |
| Nitrocellulose | 15 | g |
| N-ethyl-o,p-toluenesulfonamide | 4 | g |
| Tributyl acetyl citrate | 2 | g |
| IPA | 6.5 | g |
| qs (ethyl acetate, butyl acetate) | 100 | g |

The first composition was applied to an individual's nails or false nails. Next, after drying, the second composition was applied over the first composition. The product obtained had noteworthy staying power, including noteworthy gloss fastness.

### EXAMPLE 18

### Nail Varnish Kit

| First composition | | |
|---|---|---|
| Polymer of Example 8 | 23.8 | g AM |
| DC Red 7 Lake | 1 | g |
| Hectorite modified with | 1.3 | g |
| distearyldimethylbenzylammonium chloride (Bentone ® | | |
| 27V from Elementis) | | |
| Ethyl acetate | qs 100 | g |
| Second composition | | |
| Nitrocellulose | 15 | g |
| N-ethyl-o,p-toluenesulfonamide | 4 | g |
| Tributyl acetyl citrate | 2 | g |
| IPA | 6.5 | g |
| qs (ethyl acetate, butyl acetate) | 100 | g |

The first composition was applied to an individual's nails or false nails. Next, after drying, the second composition was applied over the first composition. The product obtained has noteworthy staying power, including noteworthy gloss fastness.

### EXAMPLE 19

### Mascara Kit

| First composition | | |
|---|---|---|
| Beeswax | 6% | |
| Paraffin wax | 13% | |
| Hydrogenated jojoba oil | 2% | |
| Water-soluble film-forming polymer | 3% | |
| Triethanolamine stearate | 8% | |
| Black pigment | 5% | |
| Preserving agent | qs | |
| Water | qs | |
| | 100% | |
| Second composition | | |
| Beeswax | 8 | g |
| Paraffin wax | 3 | g |
| Carnauba wax | 6 | g |
| Hectorite modified with | 5.3 | g |
| distearyldimethylbenzylammonium chloride (Bentone ® | | |
| 38V from Elementis) | | |
| Propylene carbonate | 1.7 | g |
| Filler | 1 | g |
| Pigments | 5 | g |
| Polymer of Example 4 | 12 | g AM |

| | |
|---|---|
| Isododecane | qs 100 |

The second composition was applied to an individual's eyelashes. Next, after drying, the first composition was applied over this second composition. The product obtained had very good staying power.

### EXAMPLE 20

### Mascara Kit

| | |
|---|---|
| First composition | |
| Beeswax | 6% |
| Paraffin wax | 13% |
| Hydrogenated jojoba oil | 2% |
| Water-soluble film-forming polymer | 3% |
| Triethanolamine stearate | 8% |
| Black pigment | 5% |
| Preserving agent | qs |
| Water | qs 100% |
| Second composition | |
| Beeswax | 8 g |
| Paraffin wax | 3 g |
| Carnauba wax | 6 g |
| Hectorite modified with | 5.3 g |
| distearyldimethylbenzylammonium chloride | |
| (Bentone ® 38V from Elementis) | |
| Propylene carbonate | 1.7 g |
| Filler | 1 g |
| Pigments | 5 g |
| Polymer of Example 7 | 12 g AM |
| Isododecane | qs 100 |

The second composition was applied to an individual's eyelashes. Next, after drying, the first composition was applied over this second composition. The product obtained had very good staying power.

### EXAMPLE 21

### Mascara as First Composition

| | |
|---|---|
| Beeswax | 8 g |
| Paraffin wax | 3 g |
| Carnauba wax | 6 g |
| Hectorite modified with | 5.3 g |
| distearyldimethylbenzylammonium chloride | |
| (Bentone ® 38V from Elementis) | |
| Propylene carbonate | 1.7 g |
| Filler | 1 g |
| Pigments | 5 g |
| Polymer of Example 7 | 12 g AM |
| Isododecane | qs 100 |

### EXAMPLES 22 to 26

### Mascara as First Composition

| | Example | | | | |
|---|---|---|---|---|---|
| | 22 | 23 | 24 | 25 | 26 |
| Polymer of Example 2 | 34.1 | | — | — | 45 |
| Polymer of Example 4 | | 34.1 | 45 | 45 | — |
| Triblock and radial block | 3.4 | 3.4 | — | — | — |
| polymers | | | | | |
| Octyldodecanol | 0.6 | 0.6 | — | 0.6 | — |
| Parleam oil | 1 | 1 | — | 1.4 | — |
| Phenyl trimethicone | 1 | 1 | — | 1.4 | — |
| PVP eicosene | 2.5 | 2.5 | — | 3.3 | — |
| Pigment | 2.5 | 2.5 | 10 | 3.3 | 10 |
| Filler | 0.5 | — | — | — | — |
| Mean gloss at 60° | — | 54.7 | 71.9 | 81.8 | 82.5 |

The gloss of the above examples was measured under the same conditions as described above.

### EXAMPLE 27

### Mascara Kit

First composition: that of Example 25

Second composition

| | |
|---|---|
| Paraffin wax | 2.3 |
| Carnauba wax | 6.6 |
| Polyolefin wax | 2.1 |
| Beeswax | 8.3 |
| Modified hectorite | 5.8 |
| Silicone candelilla wax | 1 |
| Rice starch | 1.5 |
| Vinylpyrrolidone/eicosene copolymer | 2 |
| Vinyl acetate/allyl stearate copolymer | 2.7 |
| (Mexomer PQ from the company Chimex) | |
| Polyvinyl laurate | 0.7 |
| Preserving agents | 0.1 |
| Polybutene | 1 |
| Propylene carbonate | 1.9 |
| Water | 7.6 |
| Ethanol | 2.7 |
| Black iron oxide | 4.2 |
| Isododecane | qs 100 |

A film of mascara composition of the second composition 300 micrometers thick was spread onto a glass plate.

It was left to dry for 2 hours at room temperature (25° C.).

A film of the first composition 300 micrometers thick was then spread on top.

The assembly was left to dry for 24 hours at room temperature (25° C.).

The mean gloss at 60° of the final film was then measured according to the method described above. The mean gloss of the product was 60.4.

1. A cosmetic product comprising at least one first and at least one second composition,
wherein the at least one first composition comprises, in a cosmetically acceptable organic liquid medium, at least one film-forming linear ethylenic block polymer, and
wherein the at least one second composition, which is different from the first, comprises a cosmetically acceptable medium.

2. The cosmetic product according to claim 1, wherein the at least one block polymer is free of styrene units.

3. The cosmetic product according to claim 1, wherein the at least one block polymer is non-elastomeric.

4. The cosmetic product according to claim 1, wherein the at least one block polymer is derived from aliphatic ethylenic monomers comprising a carbon-carbon double bond and at least one group chosen from ester groups and amide groups.

5. The cosmetic product according to claim 1, wherein the at least one block polymer is not soluble at an active material content of at least 1% by weight, in water or in a mixture of water and of linear or branched lower monoalcohols comprising from 2 to 5 carbon atoms, without modification of pH, at room temperature.

6. The product according to claim 1, wherein the at least one block polymer comprises at least one first and at least one second block linked together via an intermediate segment comprising at least one constituent monomer of the at least one first block and at least one constituent monomer of the at least one second block.

7. The cosmetic product according to claim 1, wherein the at least one block polymer comprises at least one first and at least one second block that have different glass transition temperatures (Tg).

8. The cosmetic product according to claim 7, wherein the at least one first and at least one second block are linked together via an intermediate segment with a glass transition temperature that is between the glass transition temperatures of the at least one first and at least one second blocks.

9. The cosmetic product according to claim 1, wherein the at least one block polymer comprises at least one first and at least one second block that are incompatible in the organic liquid medium.

10. The cosmetic product according to claim 1, wherein the at least one block polymer has a polydispersity index I of greater than 2.

11. The cosmetic product according to claim 7, wherein the at least one first block of the at least one block polymer is chosen from:
a) a block with a Tg of greater than or equal to 40° C.,
b) a block with a Tg of less than or equal to 20° C., and
c) a block with a Tg of between 20° C. and 40° C.; and
wherein the at least one second block is chosen from a category a), b) or c) different from the at least one first block.

12. The cosmetic product according to claim 11, wherein the block with a Tg of greater than or equal to 40° C. is totally or partially derived from at least one monomer whose homopolymer has a glass transition temperature of greater than or equal to 40° C.

13. The cosmetic product according to claim 12, wherein the at least one monomer whose corresponding homopolymer has a glass transition temperature of greater than or equal to 40° C. is chosen from the following monomers:
methacrylates of formula CH₂=C(CH₃)—COOR₁, wherein R₁ is chosen from linear and branched unsubstituted alkyl groups comprising from 1 to 4 carbon atoms, and C₄ to C₁₂ cycloalkyl groups,
acrylates of formula CH₂=CH—COOR₂ wherein R₂ is chosen from C₄ to C₁₂ cycloalkyl groups,
(meth)acrylamides of formula: 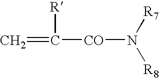
wherein R₇ and R₈, which may be identical or different, are chosen from hydrogen atoms and linear and branched C₁ to C₁₂ alkyl groups; or, alternatively, R₇ is a hydrogen atom and R₈ is a 1,1-dimethyl-3-oxobutyl group, and R′ is chosen from a hydrogen atom and a methyl group;
and mixtures thereof.

14. The cosmetic product according to claim 13, wherein R₁ is chosen from methyl, ethyl, propyl and isobutyl groups.

15. The cosmetic product according to claim 13, wherein R₂ is chosen from isobornyl acrylate and tert-butyl groups.

16. The cosmetic product according to claim 13, wherein R₇ and R₈, which may be identical or different, are chosen from n-butyl, t-butyl, isopropyl, isohexyl, isooctyl and isononyl groups.

17. The cosmetic product according to claim 13, wherein the at least one monomer whose corresponding homopolymer has a glass transition temperature of greater than or equal to 40° C. is chosen from methyl methacrylate, isobutyl (meth)acrylate, isobornyl (meth)acrylate, and mixtures thereof.

18. The cosmetic product according to claim 11, wherein the block with a Tg of less than or equal to 20° C. is totally or partially derived from at least one monomer whose homopolymer has a glass transition temperature of less than or equal to 20° C.

19. The cosmetic product according to claim 18, wherein the at least one monomer whose corresponding homopolymer has a glass transition temperature of less than or equal to 20° C. is chosen from:
acrylates of formula CH₂═CHCOOR₃,
wherein R₃ is chosen from linear and branched C₁ to C₁₂ unsubstituted alkyl groups, with the exception of tert-butyl groups, wherein at least one hetero atom chosen from O, N and S atoms is optionally intercalated into said alkyl groups,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms is optionally intercalated,
vinyl esters of formula R₅—CO—O—CH═CH₂
wherein R₅ is chosen from linear and branched C₄ to C₁₂ alkyl groups,
C₄ to C₁₂ alkyl vinyl ethers,
N—(C₄ to C₁₂)alkyl acrylamides,
and mixtures thereof.

20. The cosmetic product according to claim 19, wherein the N—(C₄ to C₁₂)alkyl acrylamides are N-octylacrylamide.

21. The cosmetic product according to claim 19, wherein the at least one monomer whose corresponding homopolymer has a glass transition temperature of less than or equal to 20° C. is chosen from alkyl acrylates whose alkyl chain comprises from 1 to 10 carbon atoms, with the proviso that the alkyl chain is not a tert-butyl group.

22. The cosmetic product according to claim 11, wherein the block with a Tg of between 20° C. and 40° C. is totally or partially derived from at least one monomer whose homopolymer has a glass transition temperature of between 20° C. and 40° C.

23. The cosmetic product according to claim 11, wherein the block with a Tg of between 20° C. and 40° C. is totally or partially derived from at least one monomer whose corresponding homopolymer has a Tg of greater than or equal to 40° C. and from at least one monomer whose corresponding homopolymer has a Tg of less than or equal to 20° C.

24. The cosmetic product according to claim 11, wherein the block with a Tg of between 20° C. and 40° C. is totally or partially derived from monomers chosen from methyl methacrylate, isobornyl acrylate, isobornyl methacrylate, butyl acrylate, 2-ethylhexyl acrylate, and mixtures thereof.

25. The cosmetic product according to claim 11, wherein the at least one first block has a Tg of greater than or equal to 40° C. and the at least one second block has a Tg of less than or equal to 20° C.

26. The cosmetic product according to claim 25, wherein the at least one first block is totally or partially derived from at least one monomer whose homopolymer has a glass transition temperature of greater than or equal to 40° C.

27. The cosmetic product according to claim 26, wherein the at least one first block is a copolymer derived from monomers whose homopolymers have a glass transition temperature of greater than or equal to 40° C.

28. The cosmetic product according to claim 26, wherein the at least one monomer whose corresponding homopolymer has a glass transition temperature of greater than or equal to 40° C. is chosen from:
methacrylates of formula CH₂═C(CH₃)—COOR₁
wherein R₁ is chosen from linear and branched unsubstituted alkyl groups comprising from 1 to 4 carbon atoms, and C₄ to C₁₂ cycloalkyl groups,
acrylates of formula CH₂═CH—COOR₂
wherein R₂ is chosen from C₄ to C₁₂ cycloalkyl groups,
(meth)acrylamides of formula: 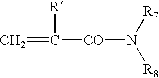
wherein R₇ and R₈, which may be identical or different, are chosen from hydrogen atoms and linear and branched C₁ to C₁₂ alkyl groups; or, alternatively, R₇ is a hydrogen atom and R₈ is 1,1-dimethyl-3-oxobutyl group, and R′ is chosen from hydrogen atoms and methyl groups;
and mixtures thereof.

29. The cosmetic product according to claim 28, wherein R₁ is chosen from methyl, ethyl, propyl and isobutyl groups.

30. The cosmetic product according to claim 28, wherein R₂ is chosen from isobornyl acrylate and tert-butyl groups.

31. The cosmetic product according to claim 28, wherein R₇ and R₈, which may be identical or different, are chosen from n-butyl, t-butyl, isopropyl, isohexyl, isooctyl and isononyl groups.

32. The cosmetic product according to claim 28, wherein the at least one monomer whose corresponding homopolymer has a glass transition temperature of greater than or equal to 40° C. is chosen from methyl methacrylate, isobutyl (meth)acrylate, isobornyl (meth)acrylate, and mixtures thereof.

33. The cosmetic product according to claim 26, wherein the at least one first block is present in an amount ranging from 20% to 90% by weight, relative to the total weight of the at least one block polymer.

34. The cosmetic product according to claim 33, wherein the at least one first block is present in an amount ranging from 30% to 80% by weight, relative to the total weight of the at least one block polymer.

35. The cosmetic product according to claim 34, wherein the at least one first block is present in an amount ranging from 50% to 70% by weight, relative to the total weight of the at least one block polymer.

36. The cosmetic product according to claim 25, wherein the at least one second block is totally or partially derived from at least one monomer whose homopolymer has a glass transition temperature of less than or equal to 20° C.

37. The cosmetic product according to claim 25, wherein the at least one second block is a homopolymer derived from at least one monomer whose homopolymer has a glass transition temperature of less than or equal to 20° C.

38. The cosmetic product according to claim 36, wherein the at least one monomer whose corresponding homopolymer has a glass transition temperature of less than or equal to 20° C. is chosen from:
acrylates of formula CH₂═CHCOOR₃,
wherein R₃ is chosen from linear and branched C₁ to C₁₂ unsubstituted alkyl groups, with the exception of tert-butyl groups, in which at least one hetero atom chosen from O, N and S atoms is optionally intercalated,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms is optionally intercalated,
vinyl esters of formula R₅—CO—O—CH═CH₂
wherein R₅ is chosen from linear and branched C₄ to C₁₂ alkyl groups,
C₄ to C₁₂ alkyl vinyl ethers,
N—(C₄ to C₁₂)alkyl acrylamides, such as N-octylacrylamide,
and mixtures thereof.

39. The cosmetic product according to claim 38, wherein the N-(C₄ to C₁₂)alkyl acrylamides are N-octylacrylamide.

40. The cosmetic product according to claim 38, wherein the at least one monomer whose corresponding homopolymer has a glass transition temperature of less than or equal to 20° C. is chosen from alkyl acrylates whose alkyl chain comprises from 1 to 10 carbon atoms, with the proviso that the alkyl chain is not a tert-butyl group.

41. The cosmetic product according to claim 25, wherein the at least one second block with a Tg of less than or equal to 20° C. is present in an amount ranging from 5% to 75% by weight, relative to the total weight of the copolymer.

42. The cosmetic product according to claim 41, wherein the at least one second block with a Tg of less than or equal to 20° C. is present in an amount ranging from 15% to 50% by weight, relative to the total weight of the at least one block polymer.

43. The cosmetic product according to claim 42, wherein the at least one second block with a Tg of less than or equal to 20° C. is present in an amount ranging from 25% to 45% by weight, relative to the total weight of the at least one block polymer.

44. The cosmetic product according to claim 11, wherein the at least one first block has a Tg of between 20° C. and 40° C. and the at least one second block has a Tg of less than or equal to 20° C. or a Tg of greater than or equal to 40° C.

45. The cosmetic product according to claim 44, wherein the at least one first block with a Tg of between 20° C. and 40° C. is totally or partially derived from at least one monomer whose homopolymer has a Tg of between 20° C. and 40° C.

46. The cosmetic product according to claim 44, wherein the at least one first block with a Tg of between 20° C. and 40° C. is a copolymer derived from monomers whose corresponding homopolymers have a Tg of greater than or equal to 40° C. and from monomers whose corresponding homopolymers have a Tg of less than or equal to 20° C.

47. The cosmetic product according to claim 44, wherein the at least one first block with a Tg of between 20° C. and 40° C. is derived from monomers chosen from methyl methacrylate, isobornyl acrylate, isobornyl methacrylate, butyl acrylate, 2-ethylhexyl acrylate, and mixtures thereof.

48. The cosmetic product according to claim 44, wherein the at least one first block with a Tg of between 20° C. and 40° C. is present in an amount ranging from 10% to 85% by weight, relative to the total weight of the at least one block polymer.

49. The cosmetic product according to claim 48, wherein the at least one first block with a Tg of between 20° C. and 40° C. is present in an amount ranging from 30% to 80% by weight, relative to the total weight of the at least one block polymer.

50. The cosmetic product according to claim 49, wherein the at least one first block with a Tg of between 20° C. and 40° C. is present in an amount ranging from 50% to 70% by weight, relative to the total weight of the at least one block polymer.

51. The cosmetic product according to claim 44, wherein the at least one second block has a Tg of greater than or equal to 40° C. and is totally or partially derived from at least one monomer whose homopolymer has a Tg of greater than or equal to 40° C.

52. The cosmetic product according to claim 51, wherein the at least one second block has a Tg of greater than or equal to 40° C. and is a homopolymer derived from monomers whose homopolymer has a glass transition temperature of greater than or equal to 40° C.

53. The cosmetic product according to claim 51, wherein the at least one monomer whose corresponding homopolymer has a Tg of greater than or equal to 40° C. is chosen from:
methacrylates of formula CH₂=C(CH₃)—COOR₁,
wherein R₁ is chosen from linear and branched unsubstituted alkyl groups comprising from 1 to 4 carbon atoms, and C₄ to C₁₂ cycloalkyl groups,
acrylates of formula CH₂═CH—COOR₂
wherein R₂ is chosen from C₄ to C₁₂ cycloalkyl groups,
(meth)acrylamides of formula: 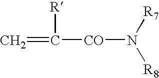
wherein R₇ and R₈, which may be identical or different, are chosen from hydrogen atoms and linear and branched C₁ to C₁₂ alkyl groups; or, alternatively, R₇ is a hydrogen atom and R₈ is a 1,1-dimethyl-3-oxobutyl group, and R′ is chosen from hydrogen atoms and methyl groups,
and mixtures thereof.

54. The cosmetic product according to claim 53, wherein R₁ is chosen from methyl, ethyl, propyl and isobutyl groups.

55. The cosmetic product according to claim 53, wherein R₂ is chosen from isobornyl acrylate and tert-butyl groups.

56. The cosmetic product according to claim 53, wherein R₇ and R₈, which may be identical or different, are chosen from n-butyl, t-butyl, isopropyl, isohexyl, isooctyl and isononyl groups.

57. The cosmetic product according to claim 53, wherein the at least one monomer whose corresponding homopolymer has a Tg of greater than or equal to 40° C. is chosen from methyl methacrylate, isobutyl methacrylate, isobornyl (meth)acrylate, and mixtures thereof.

58. The cosmetic product according to claim 51, wherein the at least one second block with a Tg of greater than or equal to 40° C. is present in an amount ranging from 10% to 85% by weight, relative to the total weight of the at least one block polymer.

59. The cosmetic product according to claim 58, wherein the at least one second block with a Tg of greater than or equal to 40° C. is present in an amount ranging from 20% to 70% by weight, relative to the total weight of the at least one block polymer.

60. The cosmetic product according to claim 59, wherein the at least one second block with a Tg of greater than or equal to 40° C. is present in an amount ranging from 30% to 70% by weight, relative to the total weight of the at least one block polymer.

61. The cosmetic product according to claim 44, wherein the at least one second block has a Tg of less than or equal to 20° C. and is totally or partially derived from at least one monomer whose homopolymer has a glass transition temperature of less than or equal to 20° C.

62. The cosmetic product according to claim 61, wherein the at least one second block has a Tg of less than or equal to 20° C. and is a homopolymer derived from monomers whose homopolymer has a glass transition temperature of less than or equal to 20° C.

63. The cosmetic product according to claim 61, wherein the at least one monomers whose corresponding homopolymer has a Tg of less than or equal to 20° C. is chosen from:
acrylates of formula CH₂═CHCOOR₃,
wherein R₃ is chosen from linear and branched C₁ to C₁₂ unsubstituted alkyl groups, with the exception of tert-buyl groups, wherein at least one hetero atom chosen from O, N and S atoms is optionally intercalated in said alkyl groups,
methacrylates of formula CH₂═C(CH₃)—COOR₄,
wherein R₄ is chosen from linear and branched C₆ to C₁₂ unsubstituted alkyl groups, in which at least one hetero atom chosen from O, N and S atoms is optionally intercalated,
vinyl esters of formula R₅—CO—O—CH=CH₂ wherein R₅ is chosen from linear and branched C₄ to C₁₂ alkyl groups,
C₄ to C₁₂ alkyl vinyl ethers,
N-(C₄ to C₁₂)alkyl acrylamides,
and mixtures thereof.

64. The cosmetic product according to claim 63, wherein the N-(C₄ to C₁₂)alkyl acrylamides are N-octylacrylamide.

65. The cosmetic product according to claim 63, wherein the at least one monomer whose homopolymers have glass transition temperatures of less than or equal to 20° C. is chosen from alkyl acrylates whose alkyl chain comprise from 1 to 10 carbon atoms, with the proviso that the alkyl chain is not a tert-butyl group.

66. The cosmetic product according to claim 61, wherein the block with a Tg of greater than or equal to 40° C. is present in an amount ranging from 20% to 90% by weight, relative to the total weight of the at least one block polymer.

67. The cosmetic product according to claim 66, wherein the block with a glass transition temperature of greater than or equal to 40° C. is present in an amount ranging from 30% to 80% by weight, relative to the total weight of the at least one block polymer.

68. The cosmetic product according to claim 67, wherein the block with a Tg of greater than or equal to 40° C. is present in an amount ranging from 50% to 70% by weight, relative to the total weight of the at least one block polymer.

69. The cosmetic product according to claim 6, wherein the at least one first block and/or the at least one second block comprises at least one additional monomer.

70. The cosmetic product according to claim 69, wherein the at least one additional monomer is chosen from hydrophilic monomers and ethylenically unsaturated monomers comprising at least one silicon atom, and mixtures thereof.

71. The cosmetic product according to claim 70, wherein the hydrophilic monomers are chosen from
ethylenically unsaturated monomers comprising at least one carboxylic or sulfonic acid functional group,
ethylenically unsaturated monomers comprising at least one tertiary amine functional group,
methacrylates of formula CH₂═C(CH₃)—COOR₆ wherein R₆ is chosen from linear and branched alkyl groups comprising from 1 to 4 carbon atoms, the alkyl group being substituted with at least one substituent chosen from hydroxyl groups and halogens,
methacrylates of formula CH₂═C(CH₃)—COOR₉, wherein R₉ is chosen from linear and branched C₆ to C₁₂ alkyl groups in which at least one hetero atom chosen from O, N and S atoms is optionally intercalated, the alkyl group being substituted with at least one substituent chosen from hydroxyl groups and halogen atoms; and
acrylates of formula CH₂═CHCOOR₁₀, wherein R₁₀ is chosen from linear and branched C₁ to C₁₂ alkyl groups substituted with at least one substituent chosen from hydroxyl groups and halogen atoms; C₁ to C₁₂ alkyl-O-polyoxyethylene groups with repetition of the oxyethylene unit ranging from 5 to 30 times; and polyoxyethylenated groups comprising from 5 to 30 ethylene oxide units.

72. The cosmetic product according to claim 71, wherein the ethylenically unsaturated monomers comprising at least one carboxylic or sulfonic acid functional group are chosen from acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof.

73. The cosmetic product according to claim 71, wherein the ethylenically unsaturated monomers comprising at least one tertiary amine function are chosen from 2-vinylpyridine, 4-vinylpyridine, dimethylaminoethyl methacrylate, diethylaminoethyl methacrylate, dimethylaminopropylmethacrylamide, and salts thereof.

74. The cosmetic product according to claim 71, wherein the alkyl group of R₆ is chosen from methyl, ethyl, propyl and isobutyl groups.

75. The cosmetic product according to claim 71, wherein the methacrylates of formula CH₂═C(CH₃)—COOR₆ are chosen from 2-hydroxypropyl methacrylate, 2-hydroxyethyl methacrylate, and trifluoroethyl methacrylate.

76. The cosmetic product according to claim 71, wherein the halogens are chosen from Cl, Br, I and F atoms.

77. The cosmetic product according to claim 71, wherein the acrylates of formula CH₂=CHCOOR₁₀ are chosen from 2-hydroxypropyl acrylate, 2-hydroxyethyl acrylate.

78. The cosmetic product according to claim 70, wherein the ethylenically unsaturated monomers comprising at least one silicon atom, are chosen from methacryloxypropyltrimethoxysilane and methacryloxypropyltris(trimethylsiloxy)silane.

79. The cosmetic product according to claim 69, wherein each of the at least one first and at least one second blocks comprises at least one additional monomer chosen from acrylic acid, (meth)acrylic acid, trifluoroethyl methacrylate, and mixtures thereof.

80. The cosmetic product according to claim 69, wherein each of the at least one first and at least one second blocks comprises at least one monomer chosen from (meth)acrylic acid esters and optionally at least one additional monomer chosen from (meth)acrylic acid, and mixtures thereof.

81. The cosmetic product according to claim 69, wherein each of the at least one first and at least one second blocks is derived totally from at least one monomer chosen from (meth)acrylic acid esters and optionally from at least one additional monomer, and mixtures thereof.

82. The cosmetic product according to claim 81, wherein the at least one additional monomer is (meth)acrylic acid.

83. The cosmetic product according to claim 69, wherein the at least one additional monomer is present in an amount ranging from 1% to 30% by weight, relative to the total weight of the first and/or second blocks.

84. The cosmetic product according to claim 7, wherein the difference between the Tg of the first and second blocks is greater than 10° C.

85. The cosmetic product according to claim 84, wherein the difference between the Tg of the first and second blocks is greater than 20° C.

86. The cosmetic product according to claim 85, wherein the difference between the Tg of the first and second blocks is greater than 30° C.

87. The cosmetic product according to claim 86, wherein the difference between the Tg of the first and second blocks is greater than 40° C.

88. The cosmetic product according to claim 10, wherein the at least one block polymer has a polydispersity index of greater than or equal to 2.5.

89. The cosmetic product according to claim 89, wherein the at least one block polymer has a polydispersity index of greater than or equal to 2.8.

90. The cosmetic product according to claim 89, wherein it has a polydispersity index ranging from 2.8 to 6.

91. The cosmetic product according to claim 1, wherein the at least one block polymer has a weight-average molecular mass (Mw) of less than or equal to 300,000.

92. The cosmetic product according to claim 91, wherein the Mw of the at least one block polymer ranges from 35,000 to 200,000.

93. The cosmetic product according to claim 92, wherein the Mw of the at least one block polymer ranges from 45,000 to 150,000.

94. The cosmetic product according to claim 1, wherein the number-average mass (Mn) of the at least one block polymer is less than or equal to 70,000.

95. The cosmetic product according to claim 94, wherein the Mn of the at least one block polymer ranges from 10,000 to 60,000.

96. The cosmetic product according to claim 95, wherein the Mn of the at least one block polymer ranges from 12,000 to 50,000.

97. The cosmetic product according to claim 1, wherein the product comprises from 0.1% to 60% by weight of polymer active material, relative to the total weight of the composition.

98. The cosmetic product according to claim 97, wherein the product comprises from 5% to 50% by weight of polymer active material, relative to the total weight of the composition.

99. The cosmetic product according to claim 98, wherein the product comprises from 10% to 40% by weight of polymer active material, relative to the total weight of the composition.

100. The cosmetic product according to claim 1, wherein the at least one second composition is transparent.

101. The cosmetic product according to claim 1, wherein the at least one second composition comprises less than 5% of pigments.

102. The cosmetic product according to claim 101, wherein the at least one second composition comprises less than 2% of pigments.

103. The cosmetic product according to claim 102, wherein the at least one second composition comprises less than 1% of pigments.

104. The cosmetic product according to claim 1, wherein the at least one first composition has a transfer index of less than 40.

105. The cosmetic product according to claim 104, wherein the at least one first composition has a transfer index of less than or equal to 30.

106. The cosmetic product according to claim 105, wherein the at least one first composition has a transfer index of less than or equal to 20.

107. The cosmetic product according to claim 106, wherein the at least one first composition has a transfer index of less than or equal to 15.

108. The cosmetic product according to claim 107, wherein the at least one first composition has a transfer index of less than or equal to 10.

109. The cosmetic product according to claim 108, wherein the at least one first composition has a transfer index of less than or equal to 5.

110. The cosmetic product according to claim 109, wherein the at least one first composition has a transfer index of less than or equal to 2 out of 100.

111. The cosmetic product according to claim 1, wherein the at least one second composition is chosen such that the transfer index of the product is less than or equal to 40.

112. The cosmetic product according to claim 111, wherein the at least one second composition is chosen such that the transfer index of the product is less than or equal to 30.

113. The cosmetic product according to claim 112, wherein the at least one second composition is chosen such that the transfer index of the product is less than or equal to 20.

114. The cosmetic product according to claim 113, wherein the at least one second composition is chosen such that the transfer index of the product is less than or equal to 15.

115. The cosmetic product according to claim 114, wherein the at least one second composition is chosen such that the transfer index of the product is less than or equal to 10.

116. The cosmetic product according to claim 115, wherein the at least one second composition is chosen such that the transfer index of the product is less than or equal to 5.

117. The cosmetic product according to claim 1, wherein the mean gloss measured at 20° C. of the at least one first composition, once spread onto a support, is greater than or equal to 30.

118. The cosmetic product according to claim 117, wherein the mean gloss measured at 20° C. of the at least one first composition, once spread onto a support, is greater than or equal to 40.

119. The cosmetic product according to claim 118, wherein the mean gloss measured at 20° C. of the at least one first composition, once spread onto a support, is greater than or equal to 50.

120. The cosmetic product according to claim 119, wherein the mean gloss measured at 20° C. of the at least one first composition, once spread onto a support, is greater than or equal to 60.

121. The cosmetic product according to claim 1, wherein the at least one second composition is chosen such that the mean gloss of the product measured at 20° C., the product being spread onto a support, is greater than or equal to 30.

122. The cosmetic product according to claim 121, wherein the at least one second composition is chosen such that the mean gloss of the product measured at 20° C., the product being spread onto a support, is greater than or equal to 40.

123. The cosmetic product according to claim 122, wherein the at least one second composition is chosen such that the mean gloss of the product measured at 20° C., the product being spread onto a support, is greater than or equal to 50.

124. The cosmetic product according to claim 123, wherein the at least one second composition is chosen such that the mean gloss of the product measured at 20° C., the product being spread onto a support, is greater than or equal to 60.

125. The cosmetic product according to claim 1, the at least one second composition is chosen such that the product, when applied to a keratin material, has at least one cosmetic property that is improved relative to the at least one first composition applied alone to the keratin material.

126. The cosmetic product according to claim 125, wherein the at least one second composition is chosen such that the product, when applied to a keratin material, is less tacky than the at least one first composition when it is applied alone to the said keratin material.

127. The cosmetic product according to claim 125, wherein the at least one second composition is chosen such that the product, when applied to a keratin material, is more comfortable than the at least one first composition when it is applied alone to the said keratin material.

128. The cosmetic product according to claim 1, wherein the cosmetically acceptable medium of the at least one second composition comprises a non-volatile liquid phase comprising at least one oil chosen from hydrocarbon-based oils, fluoro oils and silicone oils.

129. The cosmetic product according to claim 1, wherein the cosmetically acceptable medium of the at least one second composition comprises a silicone polymer with a weight-average molecular mass ranging from 200,000 to 4,000,000, chosen from dimethiconols, polydimethylsiloxanes, and mixtures thereof.

130. The cosmetic product according to claim 129, wherein the cosmetically acceptable medium of the at least one second composition comprises a silicone polymer with a weight-average molecular mass ranging from 200,000 to 2,000,000 g/mol.

131. The cosmetic product according to claim 1, wherein the at least one first composition and/or the at least one second composition comprise a coloring agent chosen from liposoluble dyes, water-soluble dyes, pigments, nacres, and mixtures thereof.

132. The cosmetic product according to claim 1, wherein the at least one first and/or the at least one second composition is in a form chosen from anhydrous liquids, anhydrous sticks, and emulsions.

133. The cosmetic product according to claim 1, wherein it is in a form chosen from foundation, makeup rouge, eyeshadow, lipstick, products with care properties, mascara, eyeliner, nail varnish, concealer products and body makeup products.

134. A cosmetic process for making up the skin and/or the lips and/or the integuments, comprising applying to the skin, the lips and/or the integuments at least one first coat of a first composition comprising, in a first cosmetically acceptable medium, at least one film-forming linear ethylenic block polymer and at least one coloring agent, and then applying, over all or part of the said first coat, at least one successive coat of a second composition comprising a second cosmetically acceptable medium.

135. A process for making up the skin and/or the lips and/or the integuments, comprising applying to the skin, the lips and/or the integuments at least one first coat of a first composition comprising, in a first cosmetically acceptable medium, at least one film-forming linear ethylenic block polymer and at least one coloring agent, leaving the at least one first coat to dry, and applying, over all or part of the at least one first coat, at least one successive coat of a second composition comprising a second cosmetically acceptable medium.

136. A process for making up the skin and/or the lips and/or the integuments, comprising applying to the skin and/or the lips and/or the integuments a cosmetic product comprising at least one first and at least one second composition, wherein the first composition comprises, in a cosmetically acceptable organic liquid medium, at least one film-forming linear ethylenic block polymer, and wherein the second composition, which is different from the first, comprises a cosmetically acceptable medium.

137. A makeup kit comprising a cosmetic product comprising at least one first and at least one second composition, wherein the first composition comprises, in a cosmetically acceptable organic liquid medium, at least one film-forming linear ethylenic block polymer, and wherein the second composition, which is different from the first, comprises a cosmetically acceptable medium.

138. The makeup kit according to claim 137, wherein the at least one first composition and the at least one second composition are packaged separately within the same container.

139. The makeup kit according to claim 137, wherein the at least one first composition and the at least one second composition are packaged in separate containers.

140. A process for making a cosmetic product for the skin and/or the lips and/or the integuments, comprising including in said cosmetic product at least one first and at least one second composition, the at least one first composition comprising, in a first cosmetically acceptable medium, at least one film-forming linear ethylenic block polymer, and the at least one second composition comprising a second cosmetically acceptable medium,
wherein at least one of the at least one first and at least one second compositions comprises a coloring agent,
wherein said at least one first and at least one second compositions are present in a combined amount such that said cosmetic product provides the skin and/or the lips and/or the integuments with a comfortable, glossy, transfer-resistant and/or migration-resistant makeup and/or a makeup with good fastness and/or with good gloss fastnessUse
